(19) United States
(12) Patent Application Publication
(10) Pub. No.: US 2025/0107990 A1
(43) Pub. Date: Apr. 3, 2025

(54) Method for Dyeing Keratinous Material Comprising the Leave-On Application of a Dye That Has a Low Water Content and Comprises C1-C6 Alkoxy Silane, Pigment and Solvent

(71) Applicant: Henkel AG & Co. KGaA, Duesseldorf (DE)

(72) Inventors: Phillip Jaiser, Langenfeld (DE); Torsten Lechner, Langenfeld (DE); Marc Nowottny, Mönchengladbach (DE); Juergen Schoepgens, Schwalmtal (DE); Avni Tairi, Antwerp (DE); Carolin Kruppa, Hilden (DE); Lea Grefe, Duesseldorf (DE); Andreas Walter, Ratingen (DE); Carsten Mathiaszyk, Essen (DE)

(21) Appl. No.: 18/980,916

(22) Filed: Dec. 13, 2024

Related U.S. Application Data
(63) Continuation of application No. PCT/EP2023/056337, filed on Mar. 13, 2023.

(30) Foreign Application Priority Data
Jun. 28, 2022 (DE) 102022206530.1

(51) Int. Cl. A61K 8/58; A61Q 5/06

(52) U.S. Cl. CPC A61K 8/585; A61Q 5/065; A61K 2800/432; A61K 2800/95

(57) ABSTRACT

The present invention relates to a method for dyeing keratinous material, in particular human hair, comprising the following steps in the specified order: (1) providing a dye (F), wherein the dye (F) in relation to the total weight of the dye (F) contains: (F1) one or more organic C₁-C₆ alkoxy silanes and/or condensation products thereof, (F2) at least one pigment, (F3) less than 25.0 wt. % water, and (F4) at least one solvent different from water, (2) administering the dye (F) to moistened or dry keratinous material, (3) optionally applying a defined amount of water to the keratinous material, which is still loaded with the dye (F), wherein the weight of the amount of water applied in step (3) is at most twice the weight of the dye (F) administered in step (2), and (4) drying the keratinous material without washing out the dye (F) beforehand.

### FIELD OF THE INVENTION

The present application relates to a method for dyeing keratinous material, in particular human hair, in which a dye that has a low water content and contains at least one C₁-C₆ alkoxy silane, a pigment and a solvent is applied to the keratinous material as a leave-on product.

### BACKGROUND OF THE INVENTION

Changing the shape and color of keratin material, in particular human hair, represents an important field of modern cosmetics. To change the hair color, the skilled artisan is familiar with a variety of coloring systems depending on the coloring requirements. Oxidation coloring agents are typically used for permanent, intense coloring with good fastness properties and good gray coverage. Such dyes contain oxidation dye precursors, so-called developer components and coupler components, which collectively form the actual dyes under the influence of oxidizing agent hydrogen peroxide. Oxidation coloring agents are characterized by very long-lasting color results.

When using direct dyes, dyes which are already formed diffuse out of the coloring agent into the hair fiber. In comparison with oxidative hair coloring, the colors obtained with direct dyes have a lower durability and a more rapid washing-out. Colors with direct dyes usually remain on the hair for a period of between 5 and 20 hair washes.

The use of color pigments is known for brief changes in color on the hair and/or the skin. Color pigments are generally understood to mean insoluble dyeing substances. These are present undissolved in the form of small particles in the coloring formulation and are only deposited from the outside onto the hair fibers and/or the skin surface. They can therefore generally be removed again without leaving residue by washing a few times with surfactant-containing cleaning agents. Various products of this type by the name of hair mascara are available on the market.

If the user desires particularly long-lasting coloring, the use of oxidative coloring agents has hitherto been the only option. However, despite multiple optimization attempts, an unpleasant ammonia odor or amine odor cannot be completely avoided in oxidative hair coloring. The hair damage that remains associated with the use of the oxidative coloring agents also has a disadvantageous effect on the hair of the user. The search for alternative, high-performance coloring methods is therefore an ongoing challenge.

EP 2168633 B1 addresses the task of producing long-lasting hair dyes using pigments. The document teaches that, when using a combination of pigment, organosilicon compound, hydrophobic polymer and a solvent, colors can be produced on hair which are particularly resistant to shampoos. 3-aminopropyl triethoxysilane was, for example, used as the organosilicon compound.

In the dyeing methods of EP 2168633 B1, organosilicon compounds from the group of silanes are used, wherein the molecular structure of these silanes comprises at least one hydroxy group and/or hydrolyzable group. Owing to the presence of the hydroxyl groups or hydrolyzable groups, the silanes are reactive substances which hydrolyze, or oligomerize, or polymerize in the presence of water. The oligomerization or polymerization of the silanes initiated by the presence of the water ultimately leads, when applied to the keratin material, to the formation of a film which fixes the dyeing compounds and thereby produces very long-lasting colors.

Upon more closely examining the dyeing methods disclosed in EP 2168633 B1 it has been found, however, that the colors produced on the hair with these agents or methods are still in need of improvement. In particular, the color intensity and the abrasion of the colors from the hair must still be improved, and also the durability, in particular the wash fastness of these colors, requires further improvement.

The object of the present invention was to find a method for dyeing keratin material such as hair, with which the pigments known from the prior art can be fixed to the hair in an extremely durable manner. The dyes should have very good storage stability, and the user should have to carry out as few steps as possible when administering the agents. In addition, the administration should be as sustainable as possible and use as little water as possible. Furthermore, the color intensity, durability and, in particular, wash fastness of the colors should be improved.

Surprisingly, it has now been found that the aforementioned objects can be achieved with excellent results if a dye (F) which has a low water content and contains one or more organic C₁-C₆ alkoxy silanes (F1), at least one pigment (F2), less than 25.0 wt. % water (F3) and at least one solvent (F4) other than water is administered to the keratin material in a dyeing method. The key aspect of the invention here is that the dye (F) is not washed out, but rather the keratin material on which the dye (F) is still present is dried. The work leading to the present invention has surprisingly shown that this dyeing method is not only more efficient and that the user consumes less water when administering the dye, but that the keratin materials dyed in this way could also be dyed more intensely and that the colors obtained had better durability, in particular better wash fastness.

### BRIEF SUMMARY OF THE INVENTION

The present invention relates to a method for dyeing keratinous material, in particular human hair, comprising the following steps in the specified order:
(1) providing a dye (F), wherein the dye (F)-based on the total weight of the dye (F)-contains:
(F1) one or more organic C₁-C₆ alkoxy silanes and/or the condensation products thereof,
(F2) at least one pigment,
(F3) less than 25.0 wt. % water, and
(F4 at least one solvent other than water,
(2) administering the dye (F) to moistened or dry keratinous material,
(3) optionally applying a specified amount of water to the keratinous material which is still coated in the dye (F), wherein the weight of the amount of water applied in step (3) is at most twice the weight of the dye (F) administered in step (2), and
(4) drying the keratinous material without washing out the dye (F) beforehand.

### DETAILED DESCRIPTION OF THE INVENTION

### Keratinous Material

Keratinous material is understood to mean hair, skin, and nails (such as fingernails and/or toenails). Furthermore, wool, furs and feathers also fall under the definition of keratinous material.

Keratinous material is preferably understood to be human hair, human skin, and human nails, in particular fingernails and toenails. Keratinous material is very particularly preferably understood to mean human hair.

### Dye (F) for Dyeing Keratinous Material

In step (1) of the method according to the invention, a dye (F) is provided. The dye (F) is characterized in that it contains-based on the total weight of the dye (F):
(F1) one or more organic C₁-C₆ alkoxy silanes and/or the condensation products thereof,
(F2) at least one pigment,
(F3) less than 25.0 wt. % water, and
(F4) at least one solvent other than water,

Within the scope of this invention, the term “coloring agent” is used for a coloring of the keratin material, in particular hair, brought about by use of pigments. In this dyeing process, the aforementioned pigments (F2) are deposited in a particularly homogeneous and smooth film on the surface of the keratin material. The film is formed in situ by oligomerization or polymerization of the organic C₁-C₆ alkoxy silane(s) (F1) and includes the pigment(s) (F2).

Organic C₁-C₆ Alkoxy Silanes (F1) and/or the Condensation Products Thereof

As the first class of substances essential to the invention, the dye (F) contains one or more organic C₁-C₆ alkoxy silanes (F1) and/or the condensation products thereof.

The organic C₁-C₆ alkoxy silanes (F1) contained in the dye (F) are reactive compounds. Organic silicon compounds, which are alternatively also referred to as organosilicon compounds, are compounds which either have a direct silicon-carbon bond (Si—C) or in which the carbon is linked to the silicon atom via an oxygen, nitrogen, or sulfur atom. The organic C₁-C₆ alkoxy silanes according to the invention are compounds containing one to three silicon atoms. Particularly preferably, the organic C₁-C₆ alkoxy silanes contain one or two silicon atoms.

According to the IUPAC rules, the designation “silane” denotes a substance group of chemical compounds based on a silicon backbone and hydrogen. In the case of organic silanes, the hydrogen atoms are replaced, completely or in part, by organic groups such as (substituted)alkyl groups and/or alkoxy groups. Some of the hydrogen atoms can also be replaced by hydroxyl groups in the organic silanes.

Organic C₁-C₆ alkoxy silanes comprising at least one C₁-C₆ alkoxy group directly bonded to the silicon atom. The alkoxy group is reactive and can first be hydrolyzed in the presence of water and then condensed with another organic C₁-C₆ alkoxy silane (or the product of the hydrolysis thereof). The C₁-C₆ alkoxy group is preferably an ethoxy group or a methoxy group. If, for example, the hydrolyzable group is an ethoxy group, the organosilicon compound preferably contains a structural unit R′R″R″Si—O—CH2—CH3. The R′, R″ and R″ functional groups here represent the three remaining free valencies of the silicon atom.

Particularly good results were obtainable when the dye (F) according to the invention contained at least one first organic C₁-C₆ alkoxy silane (F1) of formula (I).

In a particularly preferred embodiment, a method according to the invention is characterized in that the dye (F) contains at least one organic C₁-C₆ alkoxy silane (F1) of formula (I) and/or the condensation products thereof,

R₁R₂N—L—Si(OR₃)ₐ(R₄)_{b} (I),

where
R₁ and R₂ represent, independently of one another, a hydrogen atom or a C₁-C₆ alkyl group,
L represents a linear or branched divalent C₁-C₂₀ alkylene group,
R₃ and R₄ represent, independently of one another, a C₁-C₆ alkyl group,
a represents an integer from 1 to 3, and
b represents the integer 3-a.

The substituents R₁, R₂, R₃, R₄ and L in the compounds of formula (I) are explained below by way of example:

Examples of a C₁-C₆-alkyl group are the methyl, ethyl, propyl, isopropyl, n-butyl, s-butyl, and t-butyl, n-pentyl and n-hexyl groups. Propyl, ethyl, and methyl are preferred alkyl groups. Examples of a C₂-C₆ alkenyl group are vinyl, allyl, but-2-enyl, but-3-enyl and isobutenyl; preferred C₂-C₆ alkenyl groups are vinyl and allyl. Preferred examples of a hydroxy-C₁-C₆ alkyl group are a hydroxymethyl group, a 2-hydroxyethyl group, a 2-hydroxypropyl group, a 3-hydroxypropyl group, a 4-hydroxybutyl group, a 5-hydroxypentyl group and a 6-hydroxyhexyl group; a 2-hydroxyethyl group is particularly preferred. Examples of an amino-C₁-C₆ alkyl group are the aminomethyl group, the 2-aminoethyl group, the 3-aminopropyl group. The 2-aminoethyl group is particularly preferred. Examples of a linear divalent C₁-C₂₀ alkylene group are, for example, the methylene group (—CH₂—), the ethylene group (—CH₂—CH₂—), the propylene group (—CH₂—CH₂—CH₂—) and the butylene group (CH₂—CH₂—CH₂—CH₂—). The propylene group (—CH₂—CH₂—CH₂—) is particularly preferred. Starting at a chain length of 3 C atoms, divalent alkylene groups may also be branched. Examples of branched, divalent C₃-C₂₀-alkylene groups are (—CH₂—CH(CH₃)—) and (—CH₂—CH(CH₃)—CH₂—).

In the organosilicon compound of formula (I)

R₁R₂N—L—Si(OR₃)ₐ(R₄)_{b} (I),

the groups R₁ and R₂ represent, independently of one another, a hydrogen atom, or a C₁-C₆-alkyl group. Very particularly preferably, the groups R₁ and R₂ both represent a hydrogen atom.

The structural unit or the linker-L-, which represents a linear or branched, divalent C₁-C₂₀-alkylene group, is located in the middle part of the organic silicon compound.

Preferably,-L-represents a linear, divalent C₁-C₂₀ alkylene group. Further preferably,-L-represents a linear divalent C₁-C₆ alkylene group. Particularly preferably,-L-represents a methylene group (—CH₂—), an ethylene group (—CH₂—CH₂—), a propylene group (—CH₂—CH₂—CH₂—) or a butylene group (—CH₂—CH₂—CH₂—CH₂—). Very particularly preferably, L represents a propylene group (—CH₂—CH₂—CH₂—).

The organosilicon compounds of formula (I) according to the invention

R₁R₂N—L—Si(OR₃)ₐ(R₄)_{b} (I),

each bear one end of the silicon-containing grouping —Si(OR₃)ₐ(R₄)_{b}.

In the terminal structural unit-Si(OR₃)ₐ(R₄)_{b}. the functional group R₃ represents a hydrogen atom or a C₁-C₆ alkyl group, and the functional group R₄ represents a C₁-C₆ alkyl group. Particularly preferably, R₃ and R₄ represent, independently of one another, a methyl group, or an ethyl group.

In this case, a represents an integer from 1 to 3, and b represents the integer 3-a. If a represents the number 3, then b is equal to 0. If a represents the number 2, then b is equal to 1. If a represents the number 1, then b is equal to 2.

Colors with the best wash fastness were obtained when the agent according to the invention contained at least one first organosilicon compound (a1) of formula (I), where the functional groups R₃ and R₄ represent, independently of one another, a methyl group, or an ethyl group.

Furthermore, colors having the best wash fastness were obtained when the agent according to the invention contained at least one first organosilicon compound (a1) of formula (I) where the functional group a represents the number 3. In this case, the group b represents the number 0.

In another preferred embodiment, an agent according to the invention is characterized in that it contains at least one first organosilicon compound (a1) of formula (I), where
R₃, R₄ represent, independently of one another, a methyl group or an ethyl group, and
a represents the number 3, and
b represents the number 0.

In another preferred embodiment, an agent according to the invention is characterized in that it contains at least one first organosilicon compound (a1) of formula (I),

R₁R₂N—L—Si(OR₃)ₐ(R₄)_{b} (I),

where
R₁, R₂ both represent a hydrogen atom, and
L represents a linear, divalent C₁-C₆ alkylene group, preferably a propylene group (—CH₂—CH₂—CH₂—) or an ethylene group (—CH₂—CH₂—),
R₃, R₄ represent, independently of one another, a methyl group or an ethyl group, and
a represents the number 3, and
b represents the number 0.

To achieve the object according to the invention, particularly well-suited organosilicon compounds of formula (I) are:

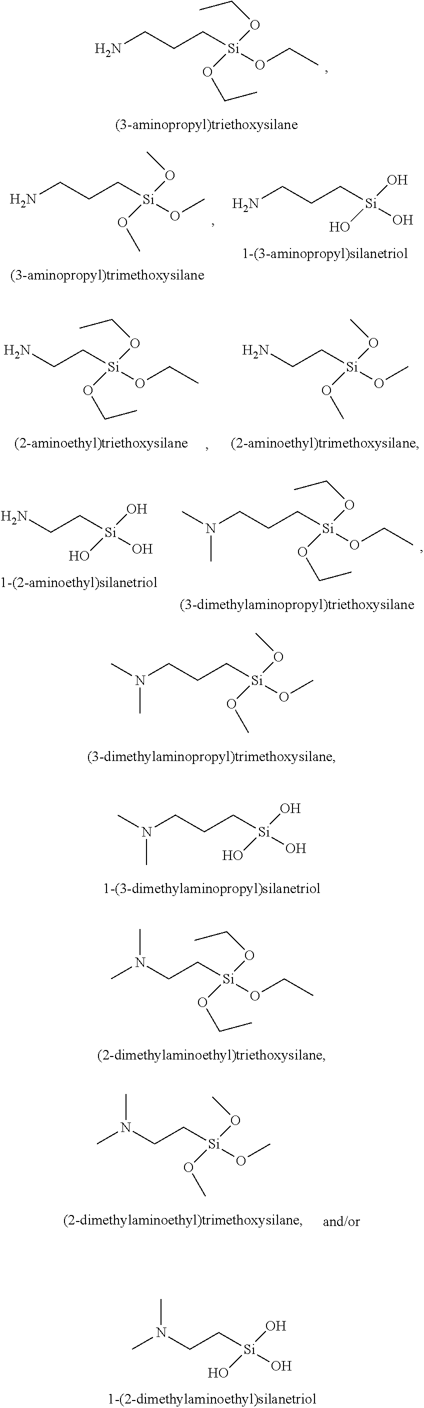

In a further preferred embodiment, a method according to the invention is characterized in that the dye (F) contains at least one organic C₁-C₆ alkoxy silane (F1) selected from the group consisting of (3-aminopropyl)trimethoxysilane (3-aminopropyl)-triethoxysilane, (2-aminoethyl)trimethoxysilane, (2-aminoethyl)triethoxysilane, (3-dimethyl-aminopropyl)-trimethoxysilane, (3-dimethylaminopropyl)triethoxysilane, (2-dimethylamino-ethyl)trimethoxysilane, (2-dimethylaminoethyl)triethoxysilane and the condensation products thereof. It is particularly preferred if the dye contains (3-aminopropyl)triethoxysilane and/or the condensation products thereof.

The aforementioned organic C₁-C₆ alkoxy silanes of formula (I) are commercially available. (3-aminopropyl)trimethoxysilane can be purchased from Sigma-Aldrich, for example. (3-aminopropyl)triethoxysilane is commercially available from Sigma-Aldrich.

In order to obtain colors with particularly good abrasion fastness and particularly high wash fastness, it has proved to be particularly advantageous if the dye (F) according to the invention contains, in addition or as an alternative to the organic C₁-C₆ alkoxy silanes of formula (I), at least one organic C₁-C₆ alkoxy silane of formula (II)

R₅Si(OR₆)ₖ(R₇)ₘ (II).

The organic C₁-C₆ alkoxysilane(s) of formula (II) can also be referred to as silanes of the alkyl alkoxysilanes type or alkyl hydroxysilanes type,

R₅Si(OR₆)ₖ(R₇)ₘ(II),

where
R₃ represents a C₁-C₁₂ alkyl group,
R₆ represents a hydrogen atom or a C₁-C₆ alkyl group,
R₇ represents a C₁-C₆ alkyl group,
k represents an integer from 1 to 3, and
m represents the integer 3-k.

In a further preferred embodiment, a method according to the invention is characterized in that the dye contains at least one organic C₁-C₆ alkoxy silane (F1) of formula (II).

R₅Si(OR₆)ₖ(R₇)ₘ (II),

where
R₃ represents a C₁-C₁₂ alkyl group,
R₆ represents a hydrogen atom or a C₁-C₆ alkyl group,
R₇ represents a C₁-C₆ alkyl group,
k represents an integer from 1 to 3, and
m represents the integer 3-k.

In a further preferred embodiment, a dye (F) used in the method according to the invention is characterized in that, in addition to the organic C₁-C₆ alkoxy silane(s) of formula (I), it contains at least one further organic C₁-C₆ alkoxy silane of formula (II)

R₅Si(OR₆)ₖ(R₇)ₘ (II),

where
R₃ represents a C₁-C₁₂ alkyl group,
R₆ represents a hydrogen atom or a C₁-C₆ alkyl group,
R₇ represents a C₁-C₆ alkyl group,
k represents an integer from 1 to 3, and
m represents the integer 3-k.

In the organic C₁-C₆ alkoxy silanes of formula (II), the group R₃ represents a C₁-C₁₂ alkyl group. This C₁-C₁₂ alkyl group is saturated and can be linear or branched. R₃ preferably represents a linear C₁-C₈ alkyl group. Preferably, R₃ represents a methyl group, an ethyl group, an n-propyl group, an n-butyl group, an n-pentyl group, an n-hexyl group, an n-octyl group or an n-dodecyl group. Particularly preferably, R₃ represents a methyl group, an ethyl group or an n-octyl group.

In the organosilicon compounds of formula (II), the functional group Re represents a hydrogen atom or a C₁-C₆ alkyl group. Particularly preferably, R₆ represents a methyl group or an ethyl group.

In the organosilicon compounds of formula (II), the functional group R₇ represents a C₁-C₆ alkyl group. Particularly preferably, R₇ represents a methyl group or an ethyl group.

Furthermore, k represents an integer from 1 to 3, and m represents the integer 3-k. If k represents the number 3, then m is equal to 0. If k represents the number 2, then m is equal to 1. If k represents the number 1, then m is equal to 2.

Colors with the best wash fastnesses were obtainable when a dye (F) containing at least one organic C₁-C₆ alkoxysilane of formula (II) in which the functional group k represents the number 3 was used in the method. In this case, the group m represents the number 0.

To achieve the object according to the invention, particularly well-suited organosilicon compounds of formula (II) are:

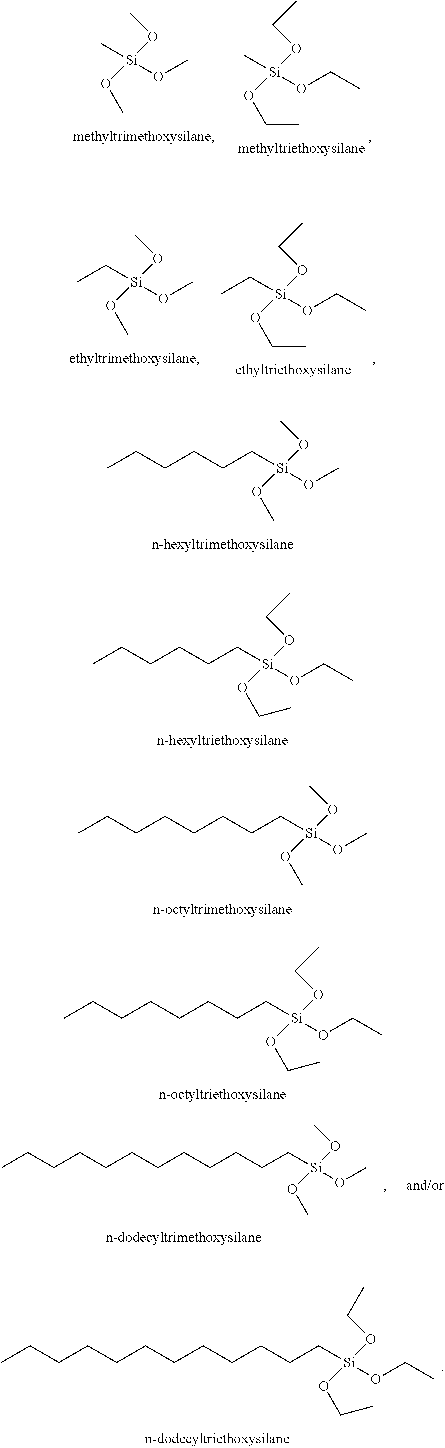

In a further preferred embodiment, a method according to the invention is characterized in that the dye (F) contains at least one organic C₁-C₆ alkoxy silane (F1) selected from the group consisting of methyltrimethoxysilane, methyltriethoxysilane, ethyltrimethoxysilane, ethyltriethoxysilane, hexyltrimethoxysilane, hexyltriethoxysilane, octyltrimethoxysilane, octyltriethoxysilane, dodecyltrimethoxysilane, dodecyltriethoxysilane and the condensation products thereof.

To achieve the object according to the invention, additional particularly well-suited organosilicon compounds are also:
vinyltrimethoxysilane and
vinyltriethoxysilane.

In an explicitly very particularly preferred embodiment, a dye (F) according to the invention is characterized in that it contains at least one first organic C₁-C₆ alkoxy silane of formula (I) selected from the group consisting of (3-aminopropyl)triethoxysilane and (3-aminopropyl)trimethoxysilane, and additionally contains at least one second organic C₁-C₆ alkoxy silane of formula (II) selected from the group consisting of methyltrimethoxy silane, methyltriethoxysilane, ethyltrimethoxysilane, ethyltriethoxysilane, hexyltrimethoxysilane and hexyltriethoxysilane.

The organic C₁-C₆ alkoxy silanes described above are reactive compounds. In order to achieve particularly good dyeing results, it is particularly advantageous to use the organic C₁-C₆ alkoxy silanes of formula (I) and/or (II) within certain quantity ranges in the dye (F).

In this context, it has been found to be preferable if the dye (F) according to the invention contains—based on the total weight of the dye-one or more organic C₁-C₆ alkoxy silanes (F1) and/or the condensation products thereof in a total amount of 0.1 to 30.0 wt. %, preferably of 0.5 to 20.0 wt. %, more preferably of 5.0 to 15.0 wt. %, and very particularly preferably of 6.0 to 12.5 wt. %.

In a further very particularly preferred embodiment, a method according to the invention is therefore characterized in that the dye (F) contains-based on the total weight of the dye (F)-one or more organic C₁-C₆ alkoxy silanes (F1) and/or the condensation products thereof in a total amount of 0.1 to 30.0 wt. %, preferably of 0.5 to 20.0 wt. %, more preferably of 5.0 to 15.0 wt. % and very particularly preferably of 6.0 to 12.5 wt. %.

### Oligomers or Condensates of Organosilicon Compounds

The organic C₁-C₆ alkoxy silanes (F1) according to the invention, in particular those of formula (I) and/or (II), are reactive compounds which can undergo a hydrolysis and condensation reaction with water.

The reaction of the organic C₁-C₆ alkoxy silanes with water can take place in various ways. The reaction starts as soon as the C₁-C₆ alkoxy silanes come into contact with water by mixing. Once C₁-C₆ alkoxy silanes and water are in contact, an exothermic hydrolysis reaction takes place according to the following scheme (reaction scheme using the example of 3-aminopropyltriethoxy silane):

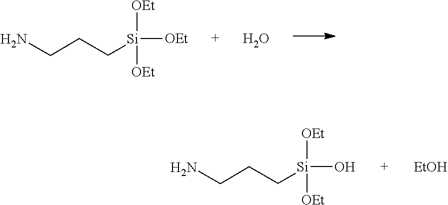

Depending on the number of hydrolyzable C₁-C₆-alkoxy groups per silane molecule, the hydrolysis reaction can also occur several times per C₁-C₆-alkoxy silane used:

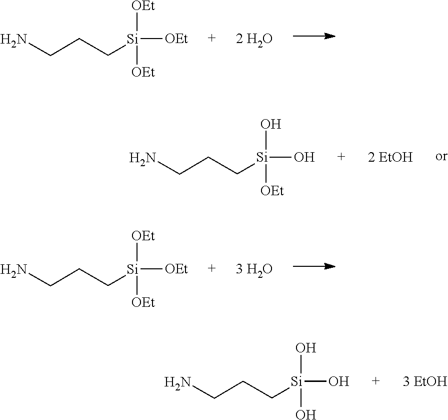

Hydrolysis using the example of methyltrimethoxysilane:

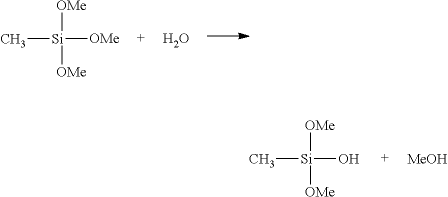

Depending on the amount of water used, the hydrolysis reaction can also take place multiple times per C₁-C₆ alkoxy silane:

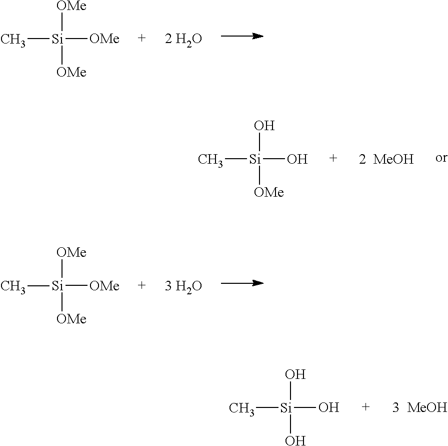

Condensation of the partially (or in parts completely) hydrolyzed C₁-C₆ alkoxy silanes takes place after hydrolysis, or almost simultaneous with hydrolysis. Precondensation can proceed, for example, according to the following scheme:

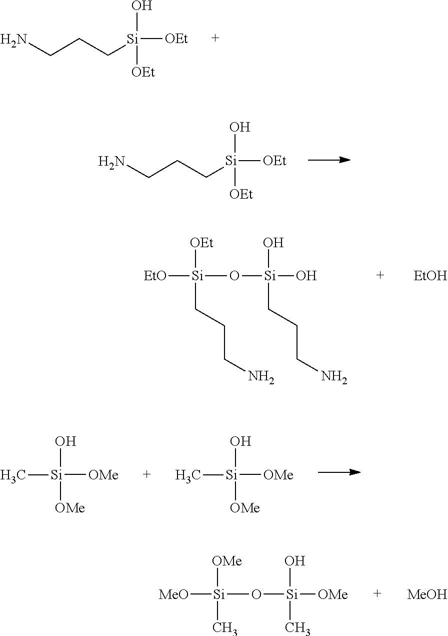

Both partially hydrolyzed and fully hydrolyzed C₁-C₆ alkoxy silanes can participate in the condensation reaction, undergoing condensation with not yet reacted, partially hydrolyzed or also fully hydrolyzed C₁-C₆ alkoxy silanes.

Possible condensation reactions are, for example (shown on the basis of the mixture (3-aminopropyl)triethoxysilane and methyltrimethoxysilane):

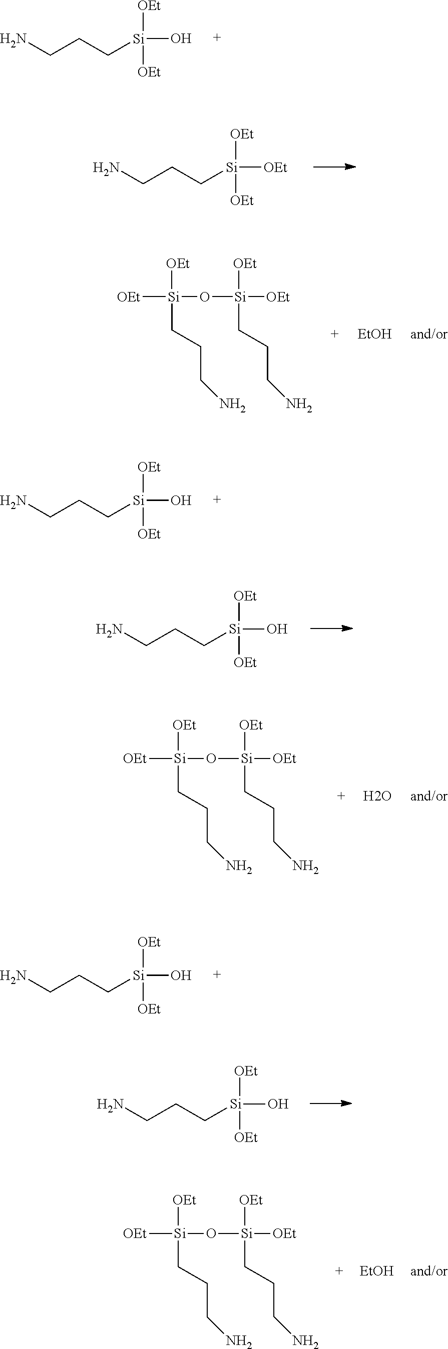

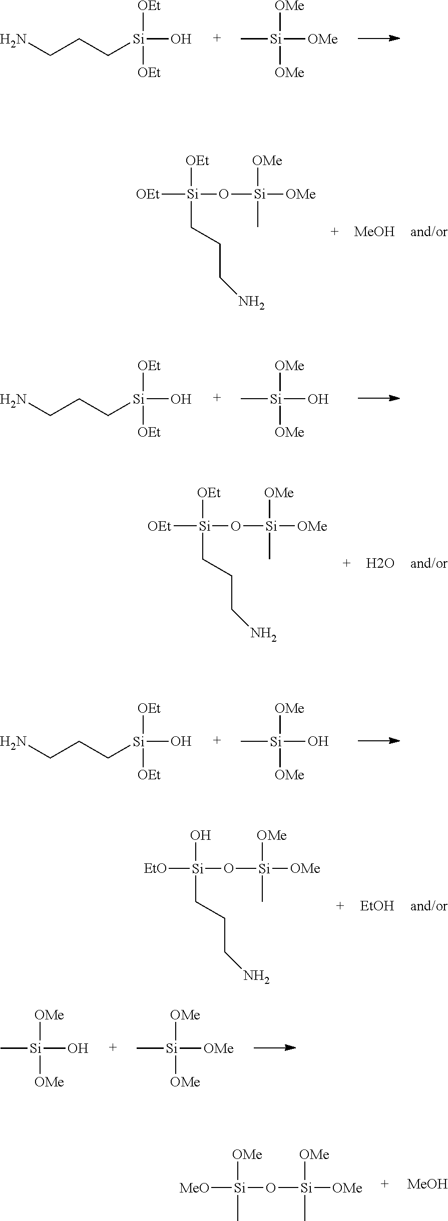

In the above reaction schemes, given by way of example, the condensation to form a dimer is shown in each case, but further condensations to oligomers having a plurality of silane atoms are also possible and preferred.

This hydrolysis or condensation reaction already starts in the presence of very low amounts of water, and therefore the oligomers and/or condensation products of the aforementioned organosilicon compounds are also included in this invention.

### Pigments (F2)

As a second essential component, the dye (F) administered in the method according to the invention contains at least one pigment (F2).

Pigments within the meaning of the present invention are understood to mean coloring compounds which have a solubility of less than 0.5 g/L, preferably of less than 0.1 g/L, even more preferably of less than 0.05 g/L, at 20° C. in water. The water solubility can be determined, for example, by means of the method described below: 0.5 g of the pigment is weighed into a beaker. A stir bar is added. Then one liter of distilled water is added. This mixture is heated to 20° C. while stirring with a magnetic stirrer for one hour. If still undissolved components of the pigment are visible in the mixture after this period, the solubility of the pigment is below 0.5 g/L. If the pigment-water mixture cannot be visually assessed due to the high intensity of the pigment that may be finely dispersed, the mixture is filtered. If a portion of undissolved pigments remains on the filter paper, the solubility of the pigment is below 0.5 g/L.

Suitable pigments or color pigments may be of inorganic and/or organic origin.

In a preferred embodiment, a method according to the invention is characterized in that the dye (F) contains at least one pigment (F2) from the group consisting of inorganic pigments, organic pigments, and/or metal pigments.

Preferred color pigments are selected from synthetic or natural inorganic pigments. Inorganic color pigments of natural origin can be produced, for example, from chalk, ocher, umbra, green soil, burnt Sienna or graphite. Furthermore, black pigments such as, for example, iron oxide black, chromatic pigments such as, for example, ultramarine or iron oxide red, and also fluorescent or phosphorescent pigments, can be used as inorganic color pigments.

Colored metal oxides, hydroxides and oxide hydrates, mixed phase pigments, sulfur-containing silicates, silicates, metal sulfides, complex metal cyanides, metal sulfates, chromates and/or molybdates are particularly suitable. Particularly preferred color pigments are black iron oxide (CI 77499), yellow iron oxide (CI 77492), red and brown iron oxide (CI 77491), manganese violet (CI 77742), ultramarine (sodium aluminum sulphosilicates, CI 77007, Pigment Blue 29), chromium oxide hydrate (CI77289), Iron Blue (ferric ferrocyanide, CI77510) and/or carmine (cochineal).

Color pigments which are likewise particularly preferred according to the invention are colored pearlescent pigments. These are usually based on mica and may be coated with one or more metal oxides. Mica is a phyllosilicate. The most important representatives of these silicates are muscovite, phlogopite, paragonite, biotite, lepidolite, and margarite. In order to produce the pearlescing pigments in conjunction with metal oxides, mica, primarily muscovite or phlogopite, is coated with a metal oxide.

As an alternative to natural mica, synthetic mica coated with one or more metal oxides(s) can also be used as a pearlescent pigment. Particularly preferred pearlescent pigments are based on natural or synthetic mica and are coated with one or more of the aforementioned metal oxides. The color of the respective pigments can be varied by varying the layer thickness of the metal oxide(s).

In another preferred embodiment, an agent according to the invention is characterized in that it contains at least one pigment selected from the group of colored metal oxides, metal hydroxides, metal oxide hydrates, silicates, metal sulfides, complex metal cyanides, metal sulphates, bronze pigments and/or from mica-based colored pigments which are coated with at least one metal oxide and/or a metal oxychloride.

In another preferred embodiment, a dye (F) according to the invention is characterized in that it contains at least one pigment (F2) selected from mica-based pigments which are coated with one or more metal oxides from the group of titanium dioxide (CI 77891), black iron oxide (CI 77499), yellow iron oxide (CI 77492), red and/or brown iron oxide (CI 77491, CI 77499), manganese violet (CI 77742), ultramarine (sodium aluminum sulfosilicates, CI 77007, pigment blue 29), chromium oxide hydrate (CI 77289), chromium oxide (CI 77288) and/or iron blue (ferric ferrocyanide, CI 77510).

Examples of particularly suitable color pigments are commercially available, for example, under the trade names Rona®, Colorona®, Xirona®, Dichrona® and Timiron® from the company Merck, Ariabel® and Unipure® from the company Sensient, Prestige® from the company Eckart Cosmetic Colors, and Sunshine® from the company Sunstar.

Very particularly preferred color pigments with the trade name Colorona® are, for example:
Colorona Copper, Merck, MICA, CI 77491 (IRON OXIDES)
Colorona Passion Orange, Merck, Mica, CI 77491 (IRON OXIDES), Alumina
Colorona Patina Silver, Merck, MICA, CI 77499 (IRON OXIDES), CI 77891 (TITANIUM DIOXIDE)
Colorona RY, Merck, CI 77891 (TITANIUM DIOXIDE), MICA, CI 75470 (CARMINE)
Colorona Oriental Beige, Merck, MICA, CI 77891 (TITANIUM DIOXIDE), CI 77491 (IRON OXIDES)
Colorona Dark Blue, Merck, MICA, TITANIUM DIOXIDE, FERRIC FERROCYANIDE
Colorona Chameleon, Merck, CI 77491 (IRON OXIDES), MICA
Colorona Aborigine Amber, Merck, MICA, CI 77499 (IRON OXIDES), CI 77891 (TITANIUM DIOXIDE)
Colorona Blackstar Blue, Merck, CI 77499 (IRON OXIDES), MICA
Colorona Patagonian Purple, Merck, MICA, CI 77491 (IRON OXIDES), CI 77891 (TITANIUM DIOXIDE), CI 77510 (FERRIC FERROCYANIDE)
Colorona Red Brown, Merck, MICA, CI 77491 (IRON OXIDES), CI 77891 (TITANIUM DIOXIDE)
Colorona Russet, Merck, CI 77491 (TITANIUM DIOXIDE), MICA, CI 77891 (IRON OXIDES)
Colorona Imperial Red, Merck, MICA, TITANIUM DIOXIDE (CI 77891), D&C RED NO. 30 (CI 73360)
Colorona Majestic Green, Merck, CI 77891 (TITANIUM DIOXIDE), MICA, CI 77288 (CHROMIUM OXIDE GREENS)
Colorona Light Blue, Merck, MICA, TITANIUM DIOXIDE (CI 77891), FERRIC FERROCYANIDE (CI 77510)
Colorona Red Gold, Merck, MICA, CI 77891 (TITANIUM DIOXIDE), CI 77491 (IRON OXIDES)
Colorona Gold Plus MP 25, Merck, MICA, TITANIUM DIOXIDE (CI 77891), IRON OXIDES (CI 77491)
Colorona Carmine Red, Merck, MICA, TITANIUM DIOXIDE, CARMINE
Colorona Blackstar Green, Merck, MICA, CI 77499 (IRON OXIDES)
Colorona Bordeaux, Merck, MICA, CI 77491 (IRON OXIDES)
Colorona Bronze, Merck, MICA, CI 77491 (IRON OXIDES)
Colorona Bronze Fine, Merck, MICA, CI 77491 (IRON OXIDES)
Colorona Fine Gold MP 20, Merck, MICA, CI 77891 (TITANIUM DIOXIDE), CI 77491 (IRON OXIDES)
Colorona Sienna Fine, Merck, CI 77491 (IRON OXIDES), MICA
Colorona Sienna, Merck, MICA, CI 77491 (IRON OXIDES)
Colorona Precious Gold, Merck, Mica, CI 77891 (Titanium dioxide), Silica, CI 77491 (IRON OXIDES), Tin oxide
Colorona Sun Gold Sparkle MP 29, Merck, MICA, TITANIUM DIOXIDE, IRON OXIDES, MICA, CI 77891, CI 77491 (EU),
Colorona Mica Black, Merck, CI 77499 (Iron oxides), Mica, CI 77891 (Titanium dioxide)
Colorona Bright Gold, Merck, Mica, CI 77891 (Titanium dioxide), CI 77491 (Iron oxides)
Colorona Blackstar Gold, Merck, MICA, CI 77499 (IRON OXIDES)

Additional particularly preferred color pigments with the trade name Xirona® are, for example:
Xirona Golden Sky, Merck, Silica, CI 77891 (Titanium Dioxide), Tin Oxide
Xirona Caribbean Blue, Merck, Mica, CI 77891 (Titanium Dioxide), Silica, Tin Oxide
Xirona Kiwi Rose, Merck, Silica, CI 77891 (Titanium Dioxide), Tin Oxide
Xirona Magic Mauve, Merck, Silica, CI 77891 (Titanium Dioxide), Tin Oxide.

In addition, particularly preferred color pigments with the trade name Unipure® are, for example:
Unipure Red LC 381 EM, Sensient CI 77491 (Iron Oxides), Silica
Unipure Black LC 989 EM, Sensient, CI 77499 (Iron Oxides), Silica
Unipure Yellow LC 182 EM, Sensient, CI 77492 (Iron Oxides), Silica

In a further embodiment, the dye (F) according to the invention can also contain one or more organic pigments.

The organic pigments according to the invention are correspondingly insoluble organic dyes or color lakes which may be selected, for example, from the group of nitroso, nitro, azo, xanthene, anthraquinone, isoindolinone, isoindoline, quinacridone, perinone, perylene, diketopyrrolopyorrole, indigo, thioindido, dioxazine, and/or triarylmethane compounds.

Particularly well suited organic pigments can for example include carmine, quinacridone, phthalocyanine, sorghum, blue pigments with the Color Index numbers CI 42090, CI 69800, CI 69825, CI 73000, CI 74100 or CI 74160, yellow pigments with the Color Index numbers CI 11680, CI 11710, CI 15985, CI 19140, CI 20040, CI 21100, CI 21108, CI 47000 or CI 47005, green pigments with the Color Index numbers CI 61565, CI 61570 or CI 74260, orange pigments with the Color Index numbers CI 11725, CI 15510, CI 45370 or CI 71105, and red pigments with the Color Index numbers CI 12085, CI 12120, CI 12370, CI 12420, CI 12490, CI 14700, CI 15525, CI 15580, CI 15620, CI 15630, CI 15800, CI 15850, CI 15865, CI 15880, CI 17200, CI 26100, CI 45380, CI 45410, CI 58000, CI 73360, CI 73915 and/or CI 75470.

In another particularly preferred embodiment, a dye (F) according to the invention is characterized in that it contains at least one organic pigment (F2) selected from the group consisting of carmine, quinacridone, phthalocyanine, sorghum, blue pigments with the Color Index numbers CI 42090, CI 69800, CI 69825, CI 73000, CI 74100 or CI 74160, yellow pigments with the Color Index numbers CI 11680, CI 11710, CI 15985, CI 19140, CI 20040, CI 21100, CI 21108, CI 47000 or CI 47005, green pigments with the Color Index numbers CI 61565, CI 61570 or CI 74260, orange pigments with the Color Index numbers CI 11725, CI 15510, CI 45370 or CI 71105, and red pigments with the Color Index numbers CI 12085, CI 12120, CI 12370, CI 12420, CI 12490, CI 14700, CI 15525, CI 15580, CI 15620, CI 15630, CI 15800, CI 15850, CI 15865, CI 15880, CI 17200, CI 26100, CI 45380, CI 45410, CI 58000, CI 73360, CI 73915 and/or CI 75470.

The organic pigment can also be a color lake. The term color lake within the meaning of the invention is understood to mean particles which comprise a layer of absorbed dyes, with the unit consisting of particles and dye being insoluble under the above-mentioned conditions. The particles may be, for example, inorganic substrates which may be aluminum, silica, calcium borosilicate, calcium aluminum borosilicate or aluminum.

For example, the alizarin color lake can be used as the color lake.

For dyeing the keratin material, pigments of a specific shape may also have been used in the dye (F). For example, a pigment based on a lamellar and/or lenticular small substrate plate may be used. Furthermore, the coloring is also possible based on a small substrate plate which comprises a vacuum-metalized pigment.

In another embodiment, a method according to the invention can be characterized in that the dye (F) also contains one or more dyeing compounds from the group of pigments based on a lamellar substrate plate, pigments based on a lenticular substrate plate, and vacuum metalized pigments.

The small substrate plates of this type have an average thickness of at most 50 nm, preferably less than 30 nm, particularly preferably at most 25 nm, for example at most 20 nm. The average thickness of the small substrate plates is at least 1 nm, preferably at least 2.5 nm, particularly preferably at least 5 nm, for example at least 10 nm. Preferred ranges for the thickness of the small substrate plates are 2.5 to 50 nm, 5 to 50 nm, 10 to 50 nm; 2.5 to 30 nm, 5 to 30 nm, 10 to 30 nm; 2.5 to 25 nm, 5 to 25 nm, 10 to 25 nm, 2.5 to 20 nm, 5 to 20 nm and 10 to 20 nm. Preferably, each substrate plate has as uniform a thickness as possible.

Due to the small thickness of the substrate plates, the pigment has a particularly high covering power.

The small substrate plates are constructed monolithically. Monolithic in this context means consisting of a single self-contained unit without fractures, stratifications, or inclusions, although structural changes may, however, occur within the substrate plates. The substrate plates are preferably composed homogeneously, i.e., there is no concentration gradient within the plates. In particular, the small substrate plates are not constructed in layers and have no particles distributed therein.

The size of the substrate plate can be matched to the respective application, in particular to the desired effect on the keratin material. In general, the small substrate plates have an average largest diameter of approximately 2 to 200 μm, in particular approximately 5 to 100 μm.

In a preferred embodiment, the form factor (aspect ratio), expressed by the ratio of the average size to the average thickness, is at least 80, preferably at least 200, more preferably at least 500, particularly preferably more than 750. In this case, the average size of the uncoated small substrate plates is understood to mean the d50 value of the uncoated small substrate plates. Unless stated otherwise, the d50 value was determined using a device of the Sympatec Helos type, having QUIXEL wet dispersion. To prepare the sample, the sample to be investigated was pre-dispersed in isopropanol for a period of 3 minutes.

The small substrate plates may be constructed from any material that can be made into the form of a small plate.

They can be of natural origin but can also be produced synthetically. Materials from which the small substrate plates can be constructed are, for example, metals and metal alloys, metal oxides, preferably aluminum oxide, inorganic compounds, and minerals such as mica and (semi-) precious stones, as well as plastics materials. Preferably, the small substrate plates are made of metal (alloy) s.

Any metal suitable for metallic luster pigments is suitable as the metal. Such metals are, inter alia, iron and steel, and all air-resistant and water-resistant (semi) metals such as, for example, platinum, zinc, chromium, molybdenum, and silicon, as well as alloys thereof such as aluminum bronzes and brass. Preferred metals are aluminum, copper, silver, and gold. Preferred small substrate plates are small aluminum plates and small brass plates, small substrate plates made of aluminum being particularly preferred.

Lamellar small substrate plates are characterized by an irregularly structured edge and are also referred to as “cornflakes” due to their appearance.

Due to their irregular structure, pigments based on lamellar small substrate plates produce a high fraction of scattered light. In addition, the pigments based on lamellar small substrate plates do not completely cover the existing color of a keratin material and, for example, effects can be achieved analogously to a natural graying.

Lenticular (=lens-shaped) small substrate plates have a substantially regular round edge and are also referred to as “silver dollars” due to their appearance. Due to their regular structure, the fraction of the reflected light predominates in the case of pigments based on lenticular small substrate plates.

Vacuum metalized pigments (VMP) can be obtained, for example, by releasing metals, metal alloys or metal oxides from correspondingly coated films. These are characterized by a particularly small thickness of the small substrate plates in the range from 5 to 50 nm and by a particularly smooth surface having increased reflectivity. Substrate plates which comprise a pigment metalized in a vacuum are also referred to as VMP substrate plates in the context of this application. VMP substrate plates of aluminum can be obtained, for example, by releasing aluminum from metalized films.

The small substrate plates made of metal or metal alloy can be passivated, for example by anodizing (oxide layer) or chromatizing.

Uncoated lamellar, lenticular and/or VPM small substrate plates, in particular those made of metal or metal alloy, reflect the incident light to a high degree and produce a light-dark flop, but no color impression.

An impression of color can be produced, for example, from optical interference effects. Such pigments can be based on at least single-coated substrate plates. These manifest interference effects by superimposing differently refracted and reflected light beams.

Accordingly, preferred pigments are pigments based on a coated small lamellar substrate plate. The substrate plate preferably has at least one coating B of a highly refractive metal oxide with a coating thickness of at least 50 nm. A coating A is preferably still between the coating B and the surface of the substrate plate. Optionally, another coating C, which is different from the underlying layer B, is on the layer B.

Suitable materials for coatings A, B and C are all substances that can be applied to the substrate plates in a film-like and permanent manner and, in the case of coatings A and B, have the required optical properties. In general, a coating of a part of the surface of the substrate plates is sufficient to obtain a pigment with a glossy effect. Thus, for example, only the upper and/or lower side of the substrate plates can be coated, the side face(s) being omitted. Preferably, the entire surface of the optionally passivated substrate plates, including the side surfaces, is covered by coating B. The substrate plates are therefore completely enveloped by coating B. This improves the optical properties of the pigment and increases the mechanical and chemical resilience of the pigments. The above also applies to layer A and preferably also to layer C if present.

Although a plurality of coatings A, B and/or C can always be present, the coated substrate plates preferably each have only one coating A, B and, if present, C.

The coating B is composed of at least one highly refractive metal oxide. Highly refractive materials have a refractive index of at least 1.9, preferably at least 2.0, and particularly preferably at least 2.4. The coating B preferably comprises at least 95 wt. %, particularly preferably at least 99 wt. %, of highly refractive metal oxide(s).

The coating B has a thickness of at least 50 nm. The thickness of coating B is preferably not more than 400 nm, particularly preferably at most 300 nm.

Highly refractive metal oxides suitable for coating B are preferably selectively light-absorbing (i.e., colored) metal oxides such as iron (III) oxide (α—and γ—Fe₂O₃, red), cobalt (II) oxide (blue), chromium (III) oxide (green), titanium (III) oxide (blue, usually in a mixture with titanium oxynitrides and titanium nitrides) and vanadium (V) oxide (orange) and mixtures thereof. Also suitable are colorless, highly-refractive oxides such as titanium dioxide and/or zirconium oxide.

Coating B can contain a selectively absorbing dye, preferably 0.001 to 5 wt. %, particularly preferably 0.01 to 1 wt. %, in each case based on the total amount of the coating B. Organic and inorganic dyes which can be stably incorporated into a metal oxide coating are suitable.

The coating A preferably has at least one low-refractive metal oxide and/or metal oxide hydrate. Preferably, coating A comprises at least 95 wt. %, particularly preferably at least 99 wt. %, low-refractive metal oxide (hydrate). Low-refractive materials have a refractive index of at most 1.8, preferably at most 1.6.

The low-refractive metal oxides suitable for coating A include, for example, silicon (di) oxide, silicon oxide hydrate, aluminum oxide, aluminum oxide hydrate, boric oxide, germanium oxide, manganese oxide, magnesium oxide and mixtures thereof, with silicon dioxide being preferred. The coating A preferably has a thickness from 1 to 100 nm, particularly preferably 5 to 50 nm, in particular preferably 5 to 20 nm.

The distance between the surface of the substrate plates and the inner surface of coating B is preferably at most 100 nm, particularly preferably at most 50 nm, in particular preferably at most 20 nm. Because the thickness of coating A and therefore the distance between the surface of the substrate plates and coating B is in the range indicated above, it can be ensured that the pigments have a high covering power.

If the pigment based on a lamellar small substrate plate has only one layer A, it is preferred for the pigment to have a lamellar small substrate plate made of aluminum and a layer A of silicon dioxide. If the pigment based on a lamellar small substrate plate has a layer A and a layer B, it is preferred for the pigment to comprise a lamellar small substrate plate made of aluminum, a layer A of silicon dioxide, and a layer B of iron oxide.

According to a preferred embodiment, the pigments have a further coating C of a metal oxide (hydrate) which is different from the underlying coating B. Suitable metal oxides are, for example, silicon (di) oxide, silicon oxide hydrate, aluminum oxide, aluminum oxide hydrate, zinc oxide, tin oxide, titanium dioxide, zirconium oxide, iron (III) oxide and chromium (III) oxide. Silicon dioxide is preferred.

The coating C preferably has a thickness of 10 to 500 nm, particularly preferably 50 to 300 nm. By providing the coating C, for example based on TiO₂, better interference can be achieved, while high covering power is maintained.

Layers A and C are in particular for corrosion protection as well as for chemical and physical stabilization. The layers A and C particularly preferably contain silicon dioxide or aluminum oxide which are applied by the sol gel method. This method comprises dispersing the uncoated lamellar substrate plates, or the lamellar substrate plates already coated with layer A and/or layer B, in a solution of a metal alkoxide such as tetraethyl orthosilicate or aluminum triisopropanolate (usually in a solution of organic solvent or a mixture of organic solvent and water with at least 50 wt. % organic solvent such as a C₁ to C₄ alcohol), and adding a weak base or acid for hydrolyzing the metal alkoxide, thereby forming a film of the metal oxide on the surface of the (coated) substrate plates.

Layer B can be produced, for example, by hydrolytic decomposition of one or more organic metal compounds and/or by precipitation of one or more dissolved metal salts and an optional subsequent post-treatment (for example, transferring formed hydroxide-containing layers into the oxide layers by tempering).

Although each of the coatings A, B and/or C can be composed of a mixture of two or more metal oxide (hydrates), each of the coatings is preferably composed of a metal oxide (hydrate).

The pigments based on coated lamellar or lenticular small substrate plates, or the pigments based on coated VMP small substrate plates preferably have a thickness of 70 to 500 nm, particularly preferably 100 to 400 nm, especially preferably 150 to 320 nm, for example 180 to 290 nm. Due to the small thickness of the substrate plates, the pigment has a particularly high covering power. The small thickness of the coated substrate plates is achieved in particular because the thickness of the uncoated substrate plates is low, but also because the thicknesses of the coatings A and, if present, C are set to the smallest possible value. The thickness of coating B determines the color impression of the pigment.

The adhesion and abrasion resistance of pigments based on coated substrate plates in the keratin material can be significantly increased by additionally modifying the outermost layer, depending on the structure of layer A, B or C, using organic compounds such as silanes, phosphoric acid esters, titanates, borates, or carboxylic acids. The organic compounds are bonded to the surface of the outermost, preferably metal oxide-containing layer A, B or C. The outermost layer denotes the layer which is spatially furthest removed from the lamellar small substrate plate. The organic compounds are preferably functional silane compounds which can bind to the metal oxide-containing layer A, B or C. These may be either monofunctional or bifunctional compounds. Examples of bifunctional organic compounds include methacryloxypropenyltrimethoxysilane, 3-methacryloxypropyltrimethoxysilane, 3-acryloxypropyltrimethoxysilane, 2-acryloxyethyltrimethoxysilane, 3-methacryloxy-propyltriethoxysilane, 3-acryloxypropyltrimethoxysilane, 2-methacryloxyethyltriethoxysilane, 2-acryloxyethyltriethoxysilane, 3-methacryloxypropyltris(methoxyethoxy) silane, 3-methacryloxypropyltris(butoxyethoxy) silane, 3-methacryloxypropyltris(propoxy) silane, 3-methacryloxypropyltris(butoxy) silane, 3-acryloxy-propyltris(methoxyethoxy) silane, 3-acryloxypropyltris(butoxyethoxy) silane, 3-acryl-oxypropyltris(butoxy) silane, vinyltrimethoxysilane, vinyltriethoxysilane, vinylethyldichlorosilane, vinylmethyldiacetoxysilane, vinylmethyldichlorosilane, vinylmethyldiethoxysilane, vinyltriacetoxysilane, vinyltrichlorosilane, phenylvinyldiethoxysilane, or phenylallyldichlorosilane. Furthermore, a modification with a monofunctional silane, in particular an alkylsilane or arylsilane, can take place. This has only one functional group, which can bind covalently to the surface of the pigment based on coated small lamellar substrate plates (i.e., to the outermost metal oxide-containing layer) or, when the covering is not entirely complete, to the metal surface. The hydrocarbon functional group of the silane faces away from the pigment. Depending on the type and nature of the hydrocarbon functional group of the silane, a different degree of hydrophobicity of the pigment is achieved. Examples of such silanes are hexadecyltrimethoxysilane, propyltrimethoxysilane, etc. Particularly preferably, pigments based on silica-coated aluminum substrate plates are surface-modified with a monofunctional silane. Octyltrimethoxysilane, octyltriethoxysilane, hexadecyltrimethoxysilane and hexadecyltriethoxysilane are particularly preferred. As a result of the altered surface properties/hydrophobization, an improvement in terms of adhesion, abrasion resistance and orientation in the application can be achieved.

Suitable pigments based on a lamellar substrate platelet include, for example, the pigments of the VISIONAIRE series by Eckart.

Pigments based on a lenticular small substrate plate are available, for example, under the name Alegrace® Gorgeous from the company Schlenk Metallic Pigments GmbH.

Pigments based on a substrate platelet, which comprises a vacuum-metalized pigment, are available, for example, under the name Alegrace® Marvelous or Alegrace® Aurous from the company Schlenk Metallic Pigments GmbH.

Owing to their excellent light and temperature resistance, the use of the aforementioned pigments in the dye (F) of the method according to the invention is very particularly preferred. It is further preferred if the pigments used have a certain particle size. It is therefore advantageous according to the invention if the at least one pigment has a mean particle size D50 from 1.0 to 50 μm, preferably from 5.0 to 45 μm, preferably from 10 to 40 μm, in particular from 14 to 30 μm. The mean particle size D50 can be determined, for example, using dynamic light scattering (DLS).

The pigment or pigments can preferably be used in an amount from 0.1 to 20.0 wt. %, preferably from 0.2 to 10.0 wt. %, in each case based on the total weight of the dye (F).

In another particularly preferred embodiment, a method according to the invention is characterized in that the dye (F) contains-based on the total weight of the dye (F)-one or more pigments (F2) in a total amount from 0.1 to 20.0 wt. %, preferably from 0.2 to 10.0 wt. %.

### Water Content of the Dye (F)

A further characteristic feature of the dye (F) is its water content (F3), which must be below 25 wt. %. In relation to its total weight, the dye (F) thus contains less than 25.0 wt. % water.

The low water content of the dye (F) ensures the storage stability of the dye (F) and also ensures that the organic C₁-C₆ alkoxysilanes are still present in reactive form and are not yet fully polymerized. If the complete crosslinking of the organic C₁-C₆ alkoxysilanes only takes place after the dye (F) has been applied to the keratin material, the film formed during crosslinking is characterized by particularly high robustness and resistance. Robust films are already obtained if the dye (F) contains less than 25.0 wt. % water. It has proven to be preferable if the water content of the dye (F) is reduced even further.

Particularly preferably, the dye (F) is manufactured with such a low water content that the water content of the dye (F)-based on the total weight of the dye (F)—is in the range of 0 to 20.0 wt. %, preferably 0.1 to 10.0 wt. %, more preferably 0.1 to 5.0 wt. %, and particularly preferably 0.5 to 3.0 wt. % water (F3).

In a further particularly preferred embodiment, a method according to the invention is characterized in that the dye (F) contains-based on the total weight of the dye (F)—from 0 to 20.0 wt. %, preferably from 0.1 to 10.0 wt. %, more preferably from 0.1 to 5.0 wt. %, and particularly preferably from 0.5 to 3.0 wt. % water (F3).

### Solvent (F4) in the Dye (F)

As a fourth component essential to the invention, the dye (F) contains at least one solvent (F4) other than water. A solvent is an established term and is used in chemistry for a substance that is liquid at room temperature (20° C.) and is capable of dissolving other chemical substances. The solvent or solvents (F4) ensure(s) fine dispersion of the pigments (F2) and provide(s) for homogeneous mixing of the pigments (F2) with the C₁-C₆ alkoxy silanes (F1). At the same time, adding at least one solvent also increases the storage stability of the dye (F).

Since the dye (F) is manufactured with a low water content, the solvent (F4) preferably forms the cosmetic carrier-either together with the water or on its own—and is thus preferably the main component of the dye (F).

Suitable solvents can be, for example, compounds from the group consisting of ethanol, isopropanol, 1,2-propylene glycol, 1,3-propylene glycol, 1,2-butylene glycol, dipropylene glycol, diethylene glycol monoethyl ether, glycerol, phenoxyethanol, benzyl alcohol, poly-C₁-C₆-alkylene glycols, dimethyl carbonate, diethyl carbonate, ethylene carbonate, propylene carbonate, butylene carbonate and glycerol carbonate.

In a further particularly preferred embodiment, a method according to the invention is characterized in that the dye (F) contains at least one solvent (F4) other than water, which is selected from the group consisting of ethanol, isopropanol, 1,2-propylene glycol 1,3-propylene glycol, ethylene glycol, 1,2-butylene glycol, dipropylene glycol, diethylene glycol monoethyl ether, glycerol, phenoxyethanol, benzyl alcohol, poly-C₁-C₆-alkylene glycols, dimethyl carbonate, diethyl carbonate, ethylene carbonate, propylene carbonate, butylene carbonate and glycerol carbonate.

In a further explicitly very particularly preferred embodiment, a method according to the invention is characterized in that the dye (F) contains at least one solvent (F4) other than water, which is selected from the group consisting of ethanol and isopropanol.

Ethanol has the CAS no. 64-17-5. Isopropanol is alternatively also referred to as 2-propanol and has the CAS no. 67-63-0. Alternatively, 1,2-propylene glycol is also referred to as 1,2-propanediol and bears the CAS numbers 57-55-6 [(RS)-1,2-dihydroxypropane], 4254-14-2 [(R)-1,2-dihydroxypropane], and 4254-153 [(S)-1,2-dihydroxypropane]. 1,3-propylene glycol is alternatively also referred to as 1,3-dihydroxypropane or 1,3-propanediol and has the CAS no. 504-63-2. Ethylene glycol is alternatively also referred to as 1,2-ethanediol and has the CAS no. 107-21-1. 1,2-butylene glycol can also be referred to as 1,2-butanediol and has the CAS numbers 584-03-2 (racemate), 40348-66-1 ((R)-enantiomer) and 73522-17-5 ((S)-enantiomer).

The dipropylene glycols (or oxydipropanols) form a group of substances derived from the glycol ether. The group of dipropylene glycols includes 2,2′-oxydi-1-propanol with CAS no. 108-61-2, 1,1′-oxydi-2-propanol with CAS no. 110-98-5 and 2-(2-hydroxypropoxy)-1-propanol with CAS no. 106-62-7. The mixture of these three isomers has the CAS no. 25265-71-8.

Diethylene glycol monoethyl ether can alternatively also be referred to as ethoxydiglycol or as ethyldiglycol or as 2-(2-ethoxyethoxy) ethanol) and has the CAS no. 111-90-0.

Glycerol is alternatively also referred to as 1,2,3-propanetriol and has the CAS number 56-81-5. Phenoxyethanol has the CAS number 122-99-6.

Benzyl alcohol can alternatively also be referred to as phenylmethanol and has the CAS no. 100-51-6.

Suitable poly-C₁-C₆-alkylene glycols include, in particular, the polyethylene glycols as described, for example, by the formula (AG)

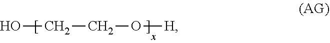

where
x represents an integer from 1 to 1000, preferably 1 to 100, particularly preferably 2 to 50.

The alkylene glycols of the formula (AG) are protic substances having at least one hydroxy group which, due to their repeating-CH₂—CH₂—O-unit, can also be referred to as polyethylene glycols insofar as x is a value of at least 2. In the alkylene glycols (α1) of formula (AG), x is an integer from 1 to 10,000. In the context of the work leading to this invention, it was found that these polyethylene glycols exhibit particularly favorable suitability for, on the one hand, improving the fastness properties of the dyeing agents and, on the other hand, also optimally adjusting the viscosity of the agents.

Polyethylene glycols with a molecular mass between 200 g/mol and 400 g/mol are non-volatile liquids at room temperature. PEG 600 has a melting range of 17 to 22° C., and therefore a pasty consistency. With molecular masses above 3000 g/mol, the PEGs are solid substances and are commercially available as flakes or powders.

A very particularly preferred low molecular weight polyethylene glycol is PEG-8, for example. PEG-8 comprises, on average, 8 ethylene glycol units (x1=8), has an average molecular weight of 400 g/mol, and bears the CAS number 25322-68-3. PEG-8 is alternatively also referred to as PEG 400 and is commercially available, for example, from APS.

Additional well-suited low molecular weight polyethylene glycols are, for example, PEG-6, PEG-7, PEG-9, and PEG-10.

Another well-suited polyethylene glycol is PEG-32, for example. PEG-32 comprises 32 ethylene glycol units (x1=32), has a mean molar mass of 1500 g/mol and bears the CAS number 25322-68-3. PEG-32 is alternatively also referred to as PEG 1500 and can, for example, be purchased commercially from Clariant.

Dimethyl carbonate is alternatively also referred to as carbonic acid dimethyl ester and has the CAS no. 616-38-6. Diethyl carbonate is alternatively also referred to as carbonic acid diethyl ester and has the CAS no. 105-58-8.

Ethylene carbonate is also referred to as 1,3-dioxolan-2-one. Ethylene carbonate corresponds to the compound of formula (I) in which R₁ and R₂ represent hydrogen and n is 0. Ethylene carbonate has the CAS number 96-49-1.

Propylene carbonate is alternatively also referred to as 4-methyl-1,3-dioxolan-2-one. Propylene carbonate corresponds to the compound of formula (I) in which R₁ is a methyl group, R₂ is hydrogen and n is the number 0. Propylene carbonate has the CAS numbers 108-32-7 [(RS)-4-methyl-1,3-dioxolan-2-one], 51260-39-0 [(S)-4-methyl-1,3-dioxolan-2-one] and 16606-55-6 [(R)-4-methyl-1,3-dioxolan-2-one]. All the aforementioned stereoisomers are covered by the invention.

According to the invention, butylene carbonate is understood to mean 1,2-butylene carbonate, which is alternatively also referred to as 4-ethyl-1,3-dioxolan-2-one and which has the CAS number 4437-85-8. Butylene carbonate corresponds to the compound of formula (I) in which R₁ is an ethyl group, R₂ is a hydrogen atom and n is the number 0.

Glycerol carbonate is alternatively also referred to as 4-hydroxymethyl-1,3-dioxolan-2-one and has the CAS number 931-40-8. Glycerol carbonate corresponds to the compound of formula (I) in which R₁ represents a hydroxymethyl group, R₂ represents a hydrogen atom, and n represents the number 0.

In a preferred embodiment, the solvent, or solvents (F4) constitute(s) the cosmetic carrier of the agent and is/are therefore preferably used as the main component in the dye (F). In this context, the main component refers to an ingredient of which the amount used exceeds that of all other ingredients.

When used as the main component, it may prove advantageous to select correspondingly high use amounts of the solvent(s) (F4). Thus, for example, the dye (F) may contain-based on the total weight of the dye (F)-one or more solvents (F4) other than water in a total amount of from 20 to 95 wt. %, preferably from 30 to 85 wt. %, more preferably from 40 to 80 wt. % and very particularly preferably from 45 to 75 wt. %.

In a further particularly preferred embodiment, a method according to the invention is characterized in that the dye (F) contains-based on the total weight of the dye (F)-one or more solvents (F4) other than water in a total amount of from 20 to 95 wt. %, preferably from 30 to 85 wt. %, more preferably from 40 to 80 wt. % and very particularly preferably from 45 to 75 wt. %.

### Fatty Components (F5) in the Dye

As a further optional component, the dye may additionally contain at least one fatty component (F5) that is liquid at 20° C. Similarly to the solvents (F4), the liquid fatty components (F5) can also ensure fine dispersion of the pigments (F2) and provide for homogeneous mixing with the silanes (F1). At the same time, adding the fatty components (F5) can further increase the storage stability of the agent.

“Fatty components” within the context of the invention are understood to be organic compounds with a solubility in water of less than 1 wt. %, and preferably less than 0.1 wt. % at room temperature (22° C.) and atmospheric pressure (760 mmHg). A fatty component liquid at 20° C. has a melting point below 20° C. (measured under atmospheric pressure (760 mmHg)).

The definition of fat constituents also explicitly includes only uncharged (i.e., non-ionic) compounds. Fat components have at least one saturated or unsaturated alkyl group having at least 8 C atoms. The molecular weight of the fat component is at most 5,000 g/mol, preferably at most 2,500 g/mol, and particularly preferably at most 1,000 g/mol. The fat components are either polyoxyalkylated or polyglycerylated compounds.

Linear or cyclic silicone oils, hydrocarbon oils, liquid fatty acid triglycerides, liquid fatty alcohols and ester oils can be mentioned as particularly suitable fatty components, provided that each compound from the aforementioned substance classes has a melting point below 20° C.

In the sense of the present invention, only non-ionic substances are explicitly considered fat components. Charged compounds, such as fatty acids and salts thereof, are not understood to be fat components.

In another particularly preferred embodiment, a method according to the invention is characterized in that the dye (F) contains at least one fatty component (F5) that is liquid at 20° C. and is selected from the group of linear or cyclic silicone oils, hydrocarbon oils, liquid fatty acid triglycerides, liquid fatty alcohols, ester oils and mixtures thereof.

Silicone oils can also be referred to as oligoalkylsiloxanes and polyalkylsiloxanes which are liquid at 20° C., i.e., the silicone oils have a melting point which is below 20° C. (at atmospheric pressure (760 mmHg)). Preferred linear silicone oils are oligoalkylsiloxanes of general formula (V):

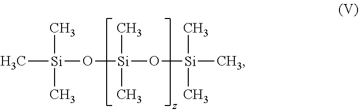

where z denotes an integer from 0 to 10,000, preferably an integer from 0 to 1,000, more preferably an integer from 0 to 100, and very particularly preferably an integer from 0 to 10.

Very particularly preferred linear oligoalkylsiloxanes are, for example:

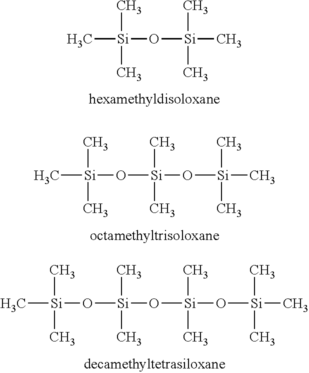

Hexamethyldisiloxane has the CAS number 107-46-0 and can be purchased commercially, for example, from Sigma-Aldrich.

Octamethyltrisiloxane has the CAS number 107-51-7 and is also commercially available from Sigma-Aldrich.

Decamethyltetrasiloxane has the CAS number 141-62-8 and is also commercially available from Sigma-Aldrich.

Another particularly well-suited silicone oil can be purchased commercially, for example, under the trade name of Dimethicone Fluid 5 cSt from the company Clearco. This silicone oil has the generic name of polydimethylsiloxane and has the CAS number 63148-62-9. The substance is a clear, colorless, and odorless liquid, low-viscosity oil.

Another silicone oil that is particularly suitable is commercially available under the trade name of Xiameter PMX 200 (1.5 cSt) from Dow Corning. This oil is also a dimethicone or polydimethylsiloxane which has the CAS number 63148-62-9.

Preferred cyclic oligoalkylsiloxanes are compounds of the general formula (VI)

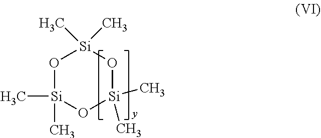

where y represents an integer from 1 to 5. Z preferably represents the numbers 1, 2 or 3.

Very particularly preferred cyclic oligoalkylsiloxanes are, for example:
hexamethylcyclotrisiloxane
octamethylcyclotetrasiloxane
decamethylcyclopentasiloxane

In another preferred embodiment, an agent according to the invention is characterized in that it contains, as the fatty component (c), at least one silicone oil of formula (V) and/or (VI),

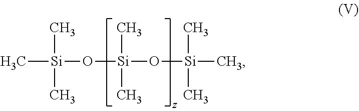

where z represents an integer from 0 to 10,000, preferably an integer from 0 to 1,000, more preferably an integer from 0 to 100, and very particularly preferably an integer from 0 to 10,

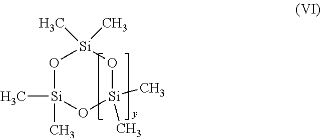

where y represents an integer from 1 to 5, preferably an integer from 1 to 3.

In another preferred embodiment, an agent according to the invention is characterized in that it contains at least one oligoalkylsiloxane (c) which is selected from the group of hexamethyldisiloxane, octamethyltrisiloxane, decamethyltetrasiloxane, hexamethylcyclotrisiloxane, octamethylcyclotetrasiloxane and/or decamethylcyclopentasiloxane.

In contrast to the reactive organosilicon compounds, in particular to the silanes of formulas (I) and (II), the oligoalkylsiloxanes are composed exclusively of dialkylsilyl groups (in particular dimethylsilyl groups) and trialkylsilyl groups (in particular trimethylsilyl groups) which are linked to one another via oxygen atoms. Thus, the oligoalkylsiloxanes within the meaning of this invention are themselves not reactive compounds and also have no hydrolyzable groups.

Hydrocarbon oils are also particularly suitable fatty components that are liquid at 20° C.

Hydrocarbons are compounds have 8 to 80 C atoms composed exclusively of carbon and hydrogen atoms. In this context, aliphatic hydrocarbons such as mineral oils, liquid paraffin oils (e.g., paraffinum liquidum or paraffinum perliquidum), isoparaffin oils and polydecenes are in particular preferred. The hydrocarbons according to the invention are also characterized in that they have a melting point which is below 20° C. under atmospheric pressure.

Liquid fatty acid triglycerides are also particularly suitable fatty components which are liquid at 20° C.

For a C₁₂-C₃₀ fatty acid triglyceride, in the context of the present invention, the triesters of trivalent alcohol glycerol are understood with three equivalents of fatty acid. Both structurally identical and different fatty acids within a triglyceride molecule can be involved in the ester formations, the premise being that the fatty acid triglyceride has a melting point below 20° C.

According to the invention, fatty acids are to be understood as saturated or unsaturated, unbranched, or branched, unsubstituted or substituted C₁₂-C₃₀ carboxylic acids. Unsaturated fatty acids may be mono-unsaturated or poly-unsaturated. With an unsaturated fatty acid, the C—C double bond(s) thereof may have the cis or trans configuration.

The fatty acid triglycerides are characterized by a particular suitability in which at least one of the ester groups, starting from glycerol, is formed with a fatty acid, which is selected from dodecanoic acid (lauric acid), tetradecanoic acid (myristic acid), hexadecanoic acid (palmitic acid), tetracosanoic acid (lignoceric acid), octadecanoic acid (stearic acid), eicosanoic acid (arachidic acid), docosanoic acid (behenic acid), petroselinic acid [(Z)-6-octadecenoic acid], palmitoleic acid [(9Z)-hexadec-9-enoic acid], oleic acid [(9Z)-octadec-9-enoic acid], elaidic acid [(9E)-octadec-9-enoic acid], erucic acid [(13Z)-docos-13-enoic acid], linoleic acid [(9Z, 12Z)-octadeca-9,12-dienoic acid, linolenic acid [(9Z, 12Z, 15Z)-octadeca-9,12, 15-trienoic acid, eleostearic acid [(9Z, 11E, 13E)-octadeca-9,11, 3-trienoic acid], arachidonic acid [(5Z, 8Z, 11Z, 14Z)-icosa-5,8,11,14-tetraenoic acid] and/or nervonic acid [(15Z)-tetracos-15-enoic acid].

Another particularly preferred embodiment is therefore an agent for dyeing keratin material which is characterized in that it contains, as a fatty component (c) that is liquid at 20° C., a naturally occurring fatty acid triglyceride and/or mixtures of naturally occurring fatty acid triglycerides which are contained in soybean oil, peanut oil, olive oil, sunflower oil, macadamia nut oil, moringa oil, apricot seed oil, marula oil and/or optionally hydrogenated castor oil.

Liquid fatty alcohols are also particularly suitable fatty components that are liquid at 20° C.

Preferred linear unsaturated fatty alcohols are (9Z)-octadec-9-en-1-ol (oleyl alcohol), (9E)-octadec-9-en-1-ol (elaidyl alcohol), (9Z,12Z)-octadeca-9,12-dien-1-ol (linoleyl alcohol), (9Z,12Z,Z)-octadeca-9,12, 15-trien-1-ol (linolenoyl alcohol), gadoleyl alcohol ((9Z)-eicos-9-en-1-ol), arachidone alcohol ((5Z,8Z,11Z,14Z)-eicosa-5,8,11,14-tetraen-1-ol), erucyl alcohol ((13Z)-docos-13-en-1-ol), and/or brassidyl alcohol ((13E)-docosen-1-ol). One example of a branched liquid fatty alcohol is 2-octyldodecanol.

Ester oils are also particularly suitable fatty components that are liquid at 20° C.

Ester oils are understood to be esters of C₁₂-C₃₀ fatty acids with aliphatic C₁-C₂₄ alcohols that have a liquid aggregate state at room temperature (20° C.). In other words, ester oils according to the invention are characterized in that they have a melting point at standard pressure (1013 mbar) which is below 20° C.

A particularly strong improvement in the hair feel was obtainable when a post-treatment agent was applied to the previously dyed hair which contained at least one ester oil from the group consisting of monoesters of C₁₂-C₂₄ fatty acids with aliphatic monovalent C₁-C₂₄ alcohols.

In another very particularly preferred embodiment method according to the invention characterized in that the post-treatment agent contains at least one fatty component (N-3) from the group of esters consisting of a C₁₂-C₃₀ fatty acid and an aliphatic monovalent C₁-C₂₄ alcohol.

C₁₂-C₂₄ fatty acids are very particularly suitable within the group of C₁₂-C₃₀ fatty acids. Examples of C₁₂-C₂₄ fatty acids which are suitable for forming the ester oils (N-3) are caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselinic acid, linoleic acid, linolenic acid, eleostearic acid, arachidic acid, gadoleic acid, behenic acid and erucic acid and technical mixtures thereof. Examples of the fatty alcohol components in the ester oils are isopropyl alcohol, caproic alcohol, caprylic alcohol, 2-ethylhexyl alcohol, capric alcohol, lauryl alcohol, isotridecyl alcohol, myristyl alcohol, cetyl alcohol, palmoleyl alcohol, stearyl alcohol, isostearyl alcohol, oleyl alcohol, elaidyl alcohol, petroselinyl alcohol, linolyl alcohol, linolenyl alcohol, eleostearyl alcohol, arachyl alcohol, gadoleyl alcohol, behenyl alcohol, erucyl alcohol and brassidyl alcohol and technical mixtures thereof.

These C₁₂-C₂₄ fatty acids are esterified by reaction with C₁-C₂₄ aliphatic alcohol, which is particularly preferably a monoalcohol, so that the esterification produces a monoester.

The aliphatic C₁-C₂₄ alcohols may be linear or branched, saturated, or mono- or polyunsaturated.

For example, an alcohol selected from the group consisting of methanol, ethanol, n-propanol, iso-propanol, n-butanol, n-pentanol, 2-ethyl-hexanol, n-hexanol, n-octanol, n-decanol and n-dodecanol can be used as the C₁-C₂₄ aliphatic saturated alcohol.

Examples of a monovalent, unsaturated, C₁-C₂₄ alcohol are oleyl alcohol (octadec-9-en-1-ol), Oleylalkohol (Octadec-9-en-1-ol) Oleylalkohol (Octadec-9-en-1-ol) palmitoleyl alcohol (cis-9-hexadecen-1-ol), elaidyl alcohol (trans-9-octadecen-1-ol), and cis-11-octadecen-1-ol.

To form the esters, the C₁₂-C₂₄ fatty acids and the C₁-C₁₂ alcohols are selected such that the ester formed by esterification from the two reactants is an ester oil, i.e., it has a melting point below 20° C. at 1013 mbar.

Some ester oils according to the invention can be used in the form of commercially available raw materials which are mixtures of the esters which are obtained from fatty acids of different chain length and/or alcohols of different chain lengths. These raw materials can have a melting range. With these raw materials, a melting point below 20° C. means that the melting process starts at a temperature below 20° C.

If, for example, an ester oil in the form of a specific raw material can be used in the agent, where this raw material has a melting range of 16 to 27° C., this raw material contains at least one ester oil with a melting point below 20° C. This ester oil is therefore according to the invention.

Particularly preferred according to the invention are 2-ethylhexyl palmitate (Cegesoft® 24), isopropyl myristate (Rilanit® IPM), isononanoic acid C16-18 alkyl ester (Cetiol® SN), stearic acid 2-ethylhexyl ester (Cetiol® 868), cetyl oleate, glycerol tricaprylate, coconut fatty alcohol caprinate/caprylate (Cetiol® LC), n-butyl stearate, oleyl erucate (Cetiol® J 600), isopropyl palmitate (Rilanit® IPP), oleyl oleate (Cetiol®), lauric acid hexyl ester (Cetiol® A), di-n-butyl adipate (Cetiol® B), cetearyl isononanoate (Cetiol® SN), and oleic acid decyl ester (Cetiol® V).

Most particularly preferably, the ester oil (N-3) is selected from the group consisting of isopropyl myristate, 2-ethylhexyl palmitate, isononanoic acid C16-18 alkyl ester, stearic acid 2-ethylhexyl ester, cetyloleate, coconut fatty alcohol caprinate, coconut fatty alcohol caprylate, n-butyl stearate, oleyl erucate, isopropyl palmitate, oleyl oleate, lauric acid hexyl ester, cetearyl isononanoates and oleic acid decyl ester.

Alternatively, isopropyl myristate is also referred to as myristic acid isopropyl ester and has the CAS number 110-27-0. Isopropyl myristate is a colorless and odorless liquid. The melting point is 0-1° C.

2-ethylhexyl palmitate is alternatively also referred to as hexadecanoic acid 2-ethylhexyl ester and has the CAS number 29806-73-3. 2-ethylhexyl palmitate is a branched, saturated ester oil of palmitic acid and ethylhexyl alcohols. 2-ethylhexyl palmitate is present at room temperature in the form of a clear, colorless liquid which has a slightly greasy odor.

Isononanoic acid C16-18 alkyl ester is alternatively also referred to as a cetearyl isononanoate; this ester bears the CAS numbers 84878-33-1 and 84878-34-2. Isononanoic acid C₁₆₋₁₈ alkyl ester is a clear, slightly yellowish liquid. At 20° C., isononanoic acid C₁₆₋₁₈ alkyl ester has a viscosity of 19-22 mPas.

Stearic acid 2-ethylhexyl ester is alternatively also termed ethylhexyl stearate and has the CAS number 91031-48-0. Stearic acid 2-ethylhexyl ester is in the form of a clear, slightly yellowish, low-viscosity oil. At 20° C., stearic acid 2-ethylhexyl ester has a viscosity of 14-16 mPas and is accordingly an oil at room temperature.

Cetyloleate has the CAS number 22393-86-8.

Coconut fatty alcohol caprylate/caprate bears the CAS number 95912-86-0. It is a mixture of C₈-C₁₀ fatty acids with C₁₂-C₁₈ fatty alcohols which exists in the form of a yellow liquid and has a melting point of 10° C.

n-butyl stearate is alternatively also referred to as stearic acid butyl ester and has the CAS numbers 85408-76-0 (C₁₆₋₁₈) and 123-95-5 (C₁₈). n-butyl stearate is a yellowish liquid and begins to melt starting at 16° C.

Oleyl erucate bears the CAS number 17673-56-2. Oleyl erucate is a yellow liquid. At 20° C., oleyl erucate has a viscosity of 40-50 mpas and is therefore an oil at room temperature.

Isopropyl palmitate is alternatively also referred to as propan-2-yl hexadecanoate and has the CAS number 142-91-6. The melting point of isopropyl palmitate is 13.5° C.

Alternatively, oleyl oleate is also termed cis-9,10-octadecenyl-cis-9,10-octadecanoate or oleic acid oleyl ester and has the CAS number 3687-45-4. Oleyl oleate is a clear, slightly yellowish oil which at 20° C. has a viscosity of 25-30 mPas and is an oil at room temperature.

Lauric acid hexyl ester is alternatively also referred to as hexyl laurate and has the CAS number 34316-64-8. Lauric acid hexyl ester is a clear, yellowish, odorless oil at room temperature. At 20° C., lauric acid hexyl ester has a viscosity of 5-7 mpas and is accordingly an oil at room temperature.

Cetearyl isononanoate is alternatively also referred to as isononanoic acid C₁₆₋₁₈ alkyl ester and has the CAS numbers 84878-33-1 and 84878-34-2. Cetearyl isononanoate is a yellowish liquid with a melting point of 16-22° C.

Oleic acid decyl ester is alternatively also referred to as decyl oleate and has the CAS number 3687-46-5. Oleic acid decyl ester is a slightly yellowish liquid which has a viscosity of 15-20 mPas at 20° C. Oleic acid decyl ester is accordingly an oil at room temperature.

Preferably, the fatty component(s) (F5) which is/are liquid at 20° C. is/are used within certain quantity ranges in the agent according to the invention. Particularly good results were obtainable when the dye (F) contained-based on the total weight of the dye (F)-one or more fatty components (c) that were liquid at 20° C. in a total amount of from 1.0 to 99 wt. %, preferably from 5.0 to 60.0 wt. %, more preferably from 10.0 to 30.0 wt. % and very particularly preferably from 15.0 to 25.0 wt. %.

In a further particularly preferred embodiment, a method according to the invention is characterized in that the dye (F) contains-based on the total weight of the dye (F)-one or more fatty components (F5) that are liquid at 20° C. in a total amount of from 1.0 to 99 wt. %, preferably from 5.0 to 60.0 wt. %, more preferably from 10.0 to 30.0 wt. % and very particularly preferably from 15.0 to 25.0 wt. %.

### Further Optional Components of the Dye (F)

In addition to the essential components (F1), (F2), (F3) and (F4) and the optional ingredient (F5), the dye (F) may optionally also contain further ingredients.

The dye (F) can thus also contain other active ingredients, auxiliaries and additives, such as cationic, non-ionic, amphoteric, zwitterionic and/or anionic surfactants, thickening polymers, film-forming polymers, structurants such as glucose, maleic acid and lactic acid, hair-conditioning compounds such as phospholipids, for example lecithin and cephalins; perfume oils, dimethyl isosorbide and cyclodextrins; active fiber structure-improving agents, in particular mono-, di-and oligosaccharides, for example glucose, galactose, fructose and lactose; dyes for coloring the product; active anti-dandruff ingredients such as piroctone olamine, zinc omadine and climbazole; amino acids and oligopeptides; animal-and/or vegetable-based protein hydrolysates, as well as in the form of their fatty acid condensation products or optionally anionically or cationically modified derivatives; light stabilizers and UV blockers; active ingredients such as panthenol, pantothenic acid, pantolactone, allantoin, pyrrolidinone carboxylic acids and their salts, and bisabolol; polyphenols, in particular hydroxycinnamic acids, 6,7-dihydroxycoumarins, hydroxybenzoic acids, catechins, tannins, leucoanthocyanidins, anthocyanidins, flavanones, flavones and flavonols; ceramides or pseudoceramides; vitamins, provitamins and vitamin precursors; plant extracts; fats and waxes such as fatty alcohols, beeswax, montan wax and paraffins; swelling and penetrating agents such as glycerol, propylene glycol monoethyl ether, carbonates, hydrogen carbonates, guanidines, ureas and primary, secondary and tertiary phosphates; opacifiers such as latex, styrene/PVP and styrene/acrylamide copolymers; pearlescent agents such as ethylene glycol mono- and distearate as well as PEG-3-distearate; and propellants such as propane-butane mixtures, N₂O, dimethyl ether, CO₂ and air.

The selection of these additional substances is made by the person skilled in the art according to the desired properties of the agents. With respect to other optional components and the employed amounts of said components, reference is made expressly to relevant manuals known to the person skilled in the art. The additional active ingredients and auxiliaries are used in the preparations according to the invention preferably always in amounts of 0.0001 to 25 wt. %, in particular of 0.0005 to 15 wt. %, relative to the total weight of the particular agent.

### Steps in the Dyeing Method

The method according to the invention comprises the following steps in the specified order:
(1) providing the dye (F) described above,
(2) administering the dye (F) to moistened or dry keratinous material,
(3) optionally applying a specified amount of water to the keratinous material which is still coated in the dye (F), wherein the weight of the amount of water applied in step (3) is at most twice the weight of the dye (F) administered in step (2), and
(4) drying the keratin material without washing out the dye (F) beforehand.

In one embodiment, the dye (F) according to the invention in its form described above can be provided directly as such and also applied to the keratin material or hair. In this form, the dye (F) according to the invention is also the ready-to-use agent and can be provided, for example, in a bottle, a container, a tube or a can. A great advantage of this form of application is the convenient and simple form of application since the user can easily remove the dye (F) from the bottle or the container in which it was provided and apply it to the keratin material. With this embodiment, mixing, shaking and/or homogenization with one or more further components or compositions is unnecessary.

Furthermore, however, it is also possible to first produce the dye (F) in or before step (1) of the method. This production can be carried out, for example, by mixing a silane blend containing the organic C₁-C₆ alkoxysilane(s) (F1) with another agent containing the pigment(s) (F2). Water (F3) and solvent (F4) may be present in the silane blend and/or in the pigment-containing agent in this case. In this embodiment, the ingredients (F1) to (F4) can be present in at least two separately manufactured containers and can be combined or mixed together during the production of the dye (F). Even if this embodiment means more effort for the user, it may nevertheless be preferred in order to increase the storage stability of the C₁-C₆ alkoxysilanes (F1), to avoid premature conglomeration between the silane (F1) and pigments (F2) or to prevent undesirable interactions between the silane (F1) and solvent (F4).

In the second step of the method according to the invention, the dye (F) is administered to moistened or dry keratinous material. The administration or application can be carried out, for example, with the aid of a small brush, a brush or a nozzle, or the user can use their gloved hand for this purpose.

In one embodiment, the dye can be applied to the dry keratin material or hair. In this case, hydrolysis, and condensation of the organic C₁-C₆ alkoxysilanes takes place due to the amount of water (F3) contained in the dye (F) itself.

Particularly good intense and durable colors were obtained when the dye (F) was applied to previously moistened or towel-dried hair. As the dye (F) itself has a low water content or is anhydrous, the additional amount of water in the hair supports the condensation of the organic C₁-C₆ alkoxysilanes directly on the surface of the keratin. In this way, a particularly even and resistant coating is formed, which tightly envelops the hair fibers and forms directly to the region where the film is also intended to be located.

Moistened or towel-dried hair is understood to be hair that has been completely wetted with water in the sink or in the shower, then squeezed and rubbed dry with a towel (e.g., for 30 seconds). Moistened or towel-dried hair is therefore no longer dripping wet, but still moist.

In a further very particularly preferred embodiment, a method according to the invention is characterized by
(2) administering the dye (F) to keratinous material that has been moistened shortly before application.

Keratinous material such as hair, which was moistened shortly before administration, was moistened within a period of at most 30 minutes, preferably at most 10 minutes before administration.

In step 3 of the method, a specified amount of water is optionally applied to the keratinous material which is still coated in the dye (F), wherein the weight of the amount of water applied in step (3) is at most twice the weight of the dye (F) administered in step (2).

Step (3) of the method according to the invention is optional and can be carried out, for example, if the dye (F) was applied to dry keratin material in step (2), or if the amount of water present in the moistened keratin material is not yet sufficient for complete crosslinking or condensation of the organic C₁-C₆ alkoxysilanes. Additional application of the specified amount of water in step (3) can thus initiate post-condensation or post-crosslinking of the organic C₁-C₆ alkoxysilanes. This post-crosslinking ensures further solidification of the film or coating.

In step (3), however, the dye (F) should not be washed out, i.e. the additional amount of water applied to the hair coated in the dye (F) should be sufficiently large that the C₁-C₆ alkoxysilanes can come into contact with a sufficient amount of water, but the dye (F) is not washed off the hair fiber or does not run off. As the work leading to this invention has shown, this is the case when the weight of the amount of water applied in step (3) is at most twice as great as the weight of the dye (F) administered in step (2).

The amount of water and the amount of dye (F) are understood here to be the amounts by weight. Thus, if 50 g of dye (F) are applied to the hair/keratin material in step (2), a maximum of 100 g of water may be applied to the hair/keratin material in step (3).

The specified amount of water can be distributed on the keratin material in step (3) and mixes with the dye (F), which is also still on the keratin material. This mixing can be assisted by massaging it in by hand or with a small brush.

It is particularly preferred if step (3) is performed. Accordingly, very particularly is a method comprising
(2) administering the dye (F) to moistened keratinous material, and
(3) applying a specified amount of water to the keratinous material which is still coated in the dye (F), wherein the total amount of water used for moistening said material in step (2) and for application in step (3) to the keratinous material is at most twice the weight of the dye (F) administered in step (2).

Also particularly preferred is a method comprising
(2) administering the dye (F) to moistened keratinous material, and
(3) applying a specified amount of water to the keratinous material which is still coated in the dye (F), wherein the total amount of water used for moistening said material in step (2) and for application in step (3) to the keratinous material is at most precisely twice the weight of the dye (F) administered in step (2).

Thus, if 50 g of dye (F) are applied to the hair/keratin material in step (2), a maximum of 50 g of water may be applied to the hair/keratin material in step (3) with at most exactly the same amount of water.

Between steps (2) and (3) there may be (if step (3) is performed) a period of a few seconds to 60 minutes, preferably of 30 seconds to 30 minutes.

After step (3), the keratin material is dried in step (4) without the dye (F) being washed out beforehand.

During drying, the water (F3) present in the dye and the solvent(s) (F4) evaporate or vaporize, and the film formed by the condensation of the C₁-C₆ alkoxysilanes sets. Drying can take place either in the air or under the influence of heat, for example with the aid of a heating hood or a hairdryer.

Between steps (3) and (4) there may be (if step (3) is performed) a period of a few seconds to 60 minutes, preferably of 30 seconds to 30 minutes. If the keratin material is dried in the air, step (4) begins immediately after step (3).

If step (3) is not performed, there may be a period of a few seconds to 60 minutes, preferably of 30 seconds to 30 minutes, between steps (2) and (4). If the keratin material is dried in the air, step (4) begins immediately after step (2).

Drying of the keratin material or hair that takes place in step (4) can be accelerated by heat treatment. Heat treatment is understood to mean that the keratin material is brought into contact with a heated appliance, or that this heated device is used on the keratin material. Furthermore, the keratin material can also be brought into contact with warm/hot air for the heat treatment. For example, a hair dryer, a blow-dryer, a thermal hood, a straightening iron, a curling iron, or an infrared lamp may be used as the appliance.

In a particularly preferred embodiment, a method according to the invention is characterized in that the heat treatment is carried out by applying a hair dryer, a blow-dryer, a thermal hood, a straightening iron, a curling iron or an infrared lamp.

It has also been found that it is preferred for the treatment temperature during the heat treatment to be between 40° C. to 210° C., preferably from 50° C. to 190° C., more preferably from 50° C. to 170° C., even more preferably from 50° C. to 150° C. and very particularly preferably from 50° C. to 100° C. In other words, it has been found to be particularly preferred if the heat treatment is carried out using an appliance which is heated to a temperature of 40° C. to 210° C., preferably 50° C. to 190° C., more preferably 50° C. to 170° C., even more preferably 50° C. to 150° C., and very particularly preferably 50° C. to 100° C.

In a further very particularly preferred embodiment, a method according to the invention is characterized by
(4) drying the keratinous material, without washing out the dye (F) beforehand, at a temperature of from 40° C. to 210° C., preferably from 45° C. to 190° C., more preferably from 45° C. to 170° C., even more preferably from 45° C. to 150° C. and very particularly preferably from 45° C. to 100° C.

For example, the keratin material or the hair can thus be treated with a blow-dryer that blows warm or hot air onto the keratin material. This air is very particularly preferably 50 to 100° C. Alternatively, the keratin material or the hair is held under an infrared lamp, which is particularly preferably set to a temperature of 50 to 100° C. For the purpose of heat treatment, hair can also be pressed between two correspondingly temperature-controlled plates of a straightening iron, it being possible for the plates to be moved simultaneously along the fiber. The plates of the straightening iron can be set, for example, to a temperature of up to 210° C.

The heat treatment can expediently be carried out until the keratin material still coated with the dye (F) has become touch dry or is completely dry.

After or before step (4), the method according to the invention can optionally also comprise a further step. For instance, further work has shown that the durability, in particular the wash fastness, of the colors obtained can be further improved if the keratinous material has been tempered in air with a high humidity after or before step (4). Contact with the moist air resulted in the film or coating produced on the keratin material becoming even more resistant. Particularly good results were obtained when the colored or colored and dried keratin materials were tempered in air having a relative humidity of 40% to 99%, preferably of 50% to 95%, more preferably of 60% to 95% and particularly preferably of 70% to 95% (measured at 20° C. and a pressure of 11013.25 hPa).

In a further embodiment, particular preference is given to a method comprising, before or after step (4), bringing the keratinous material into contact with air having a relative humidity of 40% to 99%, preferably of 50% to 95%, more preferably of 60% to 95% and particularly preferably of 70% to 95% (measured at 20° C. and a pressure of 11013.25 hPa).

Air humidity—or humidity for short-refers to the proportion of water vapor in the mixture of gases in the air. Liquid water (e.g., raindrops, fog droplets) or ice therefore do not count as air humidity. Depending on temperature and pressure, a given volume of air can only contain a certain maximum amount of water vapor. This maximum amount of water vapor in the air is referred to as the saturation amount of water vapor. The relative air humidity, which is the most common measure of air humidity, is in this case 100%. In general, relative air humidity, expressed as a percentage (%), indicates the weight ratio of the current water vapor content to the maximum possible water vapor content for the current temperature and pressure.

At normal pressure (1013.25 hPa), for example, the saturation amount of the water vapor in the air is (corresponding to 100% relative air humidity):

| | |
|---|---|
| Temperature | Saturation amount |

| | | | |
|---|---|---|---|
| −30° | C. | 0.5 | g/m³ |
| −20° | C. | 1.0 | g/m³ |
| −15° | C. | 1.4 | g/m³ |
| −10° | C. | 2.2 | g/m³ |
| −5° | C. | 3.2 | g/m³ |
| 0° | C. | 4.9 | g/m³ |
| 5° | C. | 6.8 | g/m³ |
| 10° | C. | 9.4 | g/m³ |
| 15° | C. | 12.8 | g/m³ |
| 20° | C. | 17.3 | g/m³ |
| 20° | C. | 23.1 | g/m³ |
| 30° | C. | 30.4 | g/m³ |

At a relative air humidity below 100%, the amount of water vapor in the air decreases accordingly (measured at a normal pressure of 1013.25 hPa).

| | Amount of water vapor in the air | | |
|---|---|---|---|
| | 40% relative | 60% relative | 80% relative |
| Temperature | humidity | humidity | humidity |

| | | | | | | | |
|---|---|---|---|---|---|---|---|
| −15° | C. | 0.6 | g/m³ | 0.8 | g/m³ | 1.1 | g/m³ |
| −10° | C. | 0.9 | g/m³ | 1.3 | g/m³ | 1.8 | g/m³ |
| −5° | C. | 1.3 | g/m³ | 1.9 | g/m³ | 2.5 | g/m³ |
| 0° | C. | 1.9 | g/m³ | 2.9 | g/m³ | 3.9 | g/m³ |
| 5° | C. | 2.7 | g/m³ | 4.1 | g/m³ | 5.5 | g/m³ |
| 10° | C. | 3.7 | g/m³ | 5.6 | g/m³ | 7.5 | g/m³ |
| 15° | C. | 5.1 | g/m³ | 7.7 | g/m³ | 10.3 | g/m³ |
| 20° | C. | 6.9 | g/m³ | 10.4 | g/m³ | 13.9 | g/m³ |
| 20° | C. | 9.3 | g/m³ | 13.9 | g/m³ | 18.5 | g/m³ |
| 30° | C. | 12.1 | g/m³ | 18.2 | g/m³ | 24.3 | g/m³ |

Various measuring devices for measuring air humidity are known from the prior art and commercially available.

For example, the PCE-MMK1 moisture meter from “PCE Instruments” can be used to measure relative and absolute air humidity. Air humidity can also be measured with the Bosch PTD thermal detector from “Bosch Home and Garden”.

Bringing the keratinous material into contact with air or, in other words, treating, setting, or storing it in air with a correspondingly high level of humidity can be carried out, for example, by having the person stay in a climate chamber or by using a steam hood or climate hood. Steam hoods are commercially available and widely used in the hairdressing sector.

### Examples

### 1. Formulations

### Production of the Silane Blend

In a 500-ml round-bottomed flask, 25 g ethanol (abs.) and 49.7 g methyltriethoxysilane were mixed together while stirring. This mixture was heated to 50° C. under further stirring.

6.8 g of a 1% solution of sulfuric acid in water were then added in drops over a period of approximately 5 minutes. The temperature of the reaction mixture rose to 62° C. and dropped to 55° C. after the addition was completed. Stirring continued for a further 20 minutes. Thereafter, 18.6 g (3-aminopropyl)triethoxysilane were added in drops over a period of approximately 5 minutes. After it had all been added, the mixture was stirred at 50° C. for a further 45 minutes and subsequently poured into an air-tight glass vessel.

The silane blend produced in this way was incorporated into the following dyes (F) (all amounts in wt. % unless indicated otherwise):

| Dye (F) | FI | FII | FIII | FIV |
|---|---|---|---|---|
| Silane blend | 10 | 10 | 10 | 10 |
| Unipure Red LC 3079 | 1.0 | — | — | — |
| (Pigment Red 7, CAS no. | | | | |
| 5281-04-9, CI 15850) | | | | |
| Neelikon Quinoline Yellow | — | 1.0 | — | — |
| (Pigment, CI 47005, | | | | |
| CAS no. 95193-83-2) | | | | |
| Neelikon Brilliant Blue | — | — | 1.0 | — |
| (Pigment, CI 42090, | | | | |
| CAS no. 3844-45-9) | | | | |
| Unipure Black LC 998 | — | — | — | 1.0 |
| (Sensient, CI 77499, | | | | |
| iron oxides) | | | | |
| Hexamethyldisiloxane | 20 | 20 | 20 | 20 |
| Ethanol | up | up | up | up |
| | to 100 | to 100 | to 100 | to 100 |

### 2. Application

The dyes (FI) to (FIV) were applied to hair strands (Kerling Euronatur hair, white and Kerling 9-0, length approx. 5 cm) using the following methods:

| Comparison | Invention |
|---|---|
| Wet strand with water (0.2 g water | Wet strand with water (0.2 g water |
| per 1 g hair) | per 1 g hair) |
| Apply 0.4 g of the dye (F) to the | Apply 0.4 g of the dye (F) to the |
| strand and massage in | strand and massage in |
| Again, apply 0.4 g of water per 1 | Apply another 0.4 g water per 1 g |
| g of hair and massage in | hair and massage in |
| Leave on for 5 minutes, then rinse | Leave on for 5 minutes, then |
| with water and dry the strand | dry strand at 50° C. |
| at 50° C. | without rinsing |
| Leave strand to set for 20 minutes | Leave strand to set for 20 minutes |
| at 80% relative humidity and | at 80% relative humidity and |
| 50° C. | 50° C. |

### 3. Measurement of Wash Fastness

The dye was administered as described under point 2. After drying and setting, the strands were visually assessed under a daylight lamp. 0 HW is the color result obtained directly after dyeing. To measure wash fastness, the strands were then washed 4 and 8 times respectively (4 HW, 8 HW).

For each hair wash, a commercially available shampoo (0.25 g shampoo Schauma 7 Kräuter) per 1 g of hair) was applied to the strand and massaged in with the fingers for 30 seconds. The shampoo was then rinsed out for 1 minute under running lukewarm water, and the hair strand dried. The process described above corresponds to hair washing. For each additional hair wash, the process was repeated. After the corresponding number of hair washes, the strands were visually assessed again under a daylight lamp.

| | | | | |
|---|---|---|---|---|
| Color intensity: | ++++ very high | +++ high | ++ medium | + low |

The following color results were obtained:

| | FI (Unipure Red LC 3079 | | |
|---|---|---|---|
| | (Pigment Red 7, CAS no. | | |
| | 5281-04-9, CI 15850) | | |
| | on Euronatur hair white | | |
| | 0 HW | 4 HW | 8 HW |
| Comparison (with | Intense red +++ | red +++ | red ++ |
| rinsing) | | | |
| Invention (leave-on) | Intense red ++++ | Intense red ++++ | red +++ |

| | 0 HW | 4 HW | 8 HW |
|---|---|---|---|
| FI (Unipure Red LC 3079 | | | |
| (Pigment Red 7, CAS no. | | | |
| 5281-04-9, CI 15850) on | | | |
| Kerling 9-0 | | | |
| Comparison (with rinsing) | dark reddish | dark reddish | reddish |
| | brown ++++ | brown ++++ | brown ++ |
| Invention (leave-on) | dark reddish | dark reddish | reddish |
| | brown ++++ | brown ++++ | brown ++ |
| FII (Neelikon Quinoline | | | |
| Yellow (pigment, CI | | | |
| 47005, CAS no. | | | |
| 95193-83-2)) on | | | |
| Euronatur hair white | | | |
| Comparison (with rinsing) | yellow +++ | yellow +++ | yellow ++ |
| Invention (leave-on) | bright yellow | yellow | yellow |
| | ++++ | +++ | +++ |

| | 0 HW | 4 HW | 8 HW |
|---|---|---|---|
| FII (Neelikon Quinoline | | | |
| Yellow (Pigment, | | | |
| CI 47005, CAS no. | | | |
| 95193-83-2)) on | | | |
| Kerling 9-0 | | | |
| Comparison (with | olive-yellow | olive-yellow | olive-yellow |
| rinsing) | +++ | +++ | ++ |
| Invention | olive-yellow | olive-yellow | olive-yellow |
| (leave-on) | +++ | +++ | +++ |
| FIII (Neelikon Brilliant | | | |
| Blue (pigment, CI | | | |
| 42090, CAS no. | | | |
| 3844-45-9) on | | | |
| Euronatur hair white | | | |
| Comparison (with | dark blue | blue − | faint blue − |
| rinsing) | +++ | blotchy ++ | blotchy + |
| Invention | intense dark | intense dark | dark blue |
| (leave-on) | blue ++++ | blue ++++ | +++ |

| | FIII (Neelikon Brilliant | | |
|---|---|---|---|
| | Blue (Pigment, CI 42090, | | |
| | CAS no. 3844-45-9) | | |
| | on Kerling 9-0 | | |
| | 0 HW | 4 HW | 8 HW |
| Comparison | dark blue-brown | brownish with | gray-brown |
| (with rinsing) | +++ | blue tint ++ | ++ |
| Invention | dark blue-brown | dark blue-brown | dark blue-brown |
| (leave-on) | +++ | ++ | +++ |

| | FIV (Unipure Black LC | | |
|---|---|---|---|
| | 998 (Sensient, CI 77499, | | |
| | iron oxides)) on | | |
| | Euronatur hair white | | |
| | 0 HW | 4 HW | 8 HW |
| Comparison (with | dark gray +++ | medium gray ++ | blotchy gray + |
| rinsing) | | | |
| Invention | black +++ | dark gray +++ | medium gray ++ |
| (leave-on) | | | |

| | FIV (Unipure Black LC | | |
|---|---|---|---|
| | 998 (Sensient, CI 77499, | | |
| | iron oxides) on | | |
| | Kerling 9-0 | | |
| | 0 HW | 4 HW | 8 HW |
| Comparison (with | dark brown +++ | brown ++ | beige-brown + |
| rinsing) | | | |
| Invention | dark brown + | dark brown +++ | brown ++ |
| (leave-on) | | | |

When using the dyes (FI) to (FIV) in the leave-on dyeing method according to the invention, colors with improved color intensity and improved wash fastness were obtainable on different types of hair strands.

What is claimed is:

1. A method for dyeing keratinous material, comprising, in the specified order:
(1) providing a dye (F), wherein the dye (F) comprises-based on the total weight of the dye (F):
(F1) one or more organic C₁-C₆ alkoxy silanes and/or the condensation products thereof,
(F2) at least one pigment,
(F3) less than 25.0 wt. % water, and
(F4) at least one solvent that is not water,
(2) administering the dye (F) to moistened or dry keratinous material,
(3) optionally applying a specified amount of water to the keratinous material, which is still coated in the dye (F), wherein the weight of the amount of water applied in step (3) is at most twice the weight of the dye (F) administered in step (2), and
(4) drying the keratinous material without washing out the dye (F) beforehand.

2. The method according to claim 1, wherein the dye (F) comprises at least one organic C₁-C₆ alkoxy silane (F1) of formula (I) and/or the condensation products thereof,
R₁R₂N—L—Si(OR₃)ₐ(R₄)_{b} (I),
where
R₁ and R₂ represent, independently of one another, a hydrogen atom or a C₁-C₆ alkyl group,
L represents a linear or branched divalent C₁-C₂₀ alkylene group,
R₃ and R₄ represent, independently of one another, a C₁-C₆ alkyl group,
a represents an integer from 1 to 3, and
b represents the integer 3-a.

3. The method according to claim 1, wherein the dye (F) comprises at least one organic C₁-C₆ alkoxy silane (F1) selected from the group consisting: of (3-aminopropyl)trimethoxysilane, (3-aminopropyl)-triethoxysilane, (2-aminoethyl)trimethoxysilane, (2-aminoethyl)triethoxysilane, (3-dimethyl-aminopropyl)trimethoxysilane, (3-dimethylaminopropyl)triethoxysilane, (2-dimethylaminoethyl)trimethoxysilane, (2-dimethylaminoethyl)triethoxysilane and the condensation products thereof.

4. The method according to claim 1, wherein the dye comprises at least one organic C₁-C₆ alkoxy silane (F1) of formula (II)
R₅Si(OR₆)ₖ(R₇)ₘ (II),
where
R₃ represents a C₁-C₁₂ alkyl group,
R₆ represents a hydrogen atom or a C₁-C₆ alkyl group,
R₇ represents a C₁-C₆ alkyl group,
k represents an integer from 1 to 3, and
m represents the integer 3-k.

5. The method according to claim 1, wherein the dye (F) comprises at least one organic C₁-C₆ alkoxy silane (F1) selected from the group consisting of: methyltrimethoxysilane, methyltriethoxysilane, ethyltrimethoxysilane, ethyltriethoxysilane, hexyltrimethoxysilane, hexyltriethoxysilane, octyltrimethoxysilane, octyltriethoxysilane, dodecyltrimethoxysilane, dodecyltriethoxysilane and the condensation products thereof.

6. The method according to claim 1, wherein the dye (F) comprises, based on the total weight of the dye (F), one or more organic C₁-C₆ alkoxy silanes (F1) and/or the condensation products thereof in a total amount of 0.1 to 30.0 wt. %.

7. The method according to claim 1, wherein the dye (F) comprises, based on the total weight of the dye (F), one or more organic C₁-C₆ alkoxy silanes (F1) and/or the condensation products thereof in a total amount of 6.0 to 12.5 wt. %.

8. The agent according to claim 1, wherein the dye (F) comprises at least one pigment (F2) selected from the group consisting of: inorganic pigments, organic pigments and metal pigments.

9. The method according to claim 1, wherein the dye (F) comprises, based on the total weight of the dye (F), 0 to 20.0 wt. % water (F3).

10. The method according to claim 1, wherein the dye (F) comprises, based on the total weight of the dye (F), 0.5 to 3.0 wt. %, water (F3).

11. The method according to claim 1, wherein the dye (F) comprises at least one solvent (F4) other than water, which is selected from the group consisting of: ethanol, isopropanol, 1,2-propylene glycol, 1,3-propylene glycol, ethylene glycol, 1,2-butylene glycol, dipropylene glycol, diethylene glycol monoethyl ether, glycerol, phenoxyethanol, benzyl alcohol, poly-C₁-C₆ alkylene glycols, dimethyl carbonate, diethyl carbonate, ethylene carbonate, propylene carbonate, butylene carbonate, and glycerol carbonate.

12. The method according to claim 1, wherein the dye (F) comprises, based on the total weight of the dye (F), one or more solvents (F4) other than water in a total amount of from 20 to 95 wt. %.

13. The method according to claim 1, wherein the dye (F) comprises, based on the total weight of the dye (F), one or more solvents (F4) other than water in a total amount of from 45 to 75 wt. %.

14. The method according to claim 1, wherein the dye (F) further comprises at least one fatty component (F5) that is liquid at 20° C., said fatty component (F5) being selected from the group consisting of: linear or cyclic silicone oils, hydrocarbon oils, liquid fatty acid triglycerides, liquid fatty alcohols, ester oils and mixtures thereof.

15. The method according to claim 1, wherein the dye (F) comprises, based on the total weight of the dye (F), one or more fatty components (F5) that are liquid at 20° C. in a total amount of from 1.0 to 99 wt. %.

16. The method according to claim 1, wherein the dye (F) comprises, based on the total weight of the dye (F), one or more fatty components (F5) that are liquid at 20° C. in a total amount of from 15.0 to 25.0 wt. %.

17. The method according to claim 1, wherein shortly before administering the dye (F) to the keratinous material, the keratinous material is moistened.

18. The method according to claim 1, comprising:
(2) administering the dye (F) to moistened keratinous material, and
(3) applying a specified amount of water to the keratinous material, which is still coated in the dye (F), wherein the total amount of water used for moistening the keratinous material in step (2) and for application thereto in step (3) is at most twice the weight of the dye (F) administered in step (2).

19. The method according to claim 1, comprising:
(4) drying the keratinous material, without washing out the dye (F) beforehand, said drying occurring at a temperature of 40° C. to 210° C.

20. The method according to claim 1, comprising, before or after step (4), bringing the keratinous material into contact with air having a relative humidity of 40% to 99% (measured at 20° C. and a pressure of 11013.25 hPa).
